(19) United States
(12) Patent Application Publication
(10) Pub. No.: US 2011/0236381 A1
(43) Pub. Date: Sep. 29, 2011

(54) INHIBITION OF INTER-ALPHA TRYPSIN INHIBITOR FOR THE TREATMENT OF AIRWAY DISEASE

(76) Inventors: Stavros Garantziotis, Durham, NC (US); Yow-Pin Lim, East Providence, RI (US); John W. Hollingsworth, Durham, NC (US); W. Michael Foster, Durham, NC (US)

(21) Appl. No.: 13/133,000

(22) PCT Filed: Apr. 1, 2009
(86) PCT No.: PCT/US2009/039157

§ 371 Date: Jun. 6, 2011

Related U.S. Application Data
(60) Provisional application No. 61/121,407, filed on Dec. 10, 2008.

(51) Int. Cl. A61K 39/395; A61K 31/7088; A61K 38/02; A61P 11/06; A61P 11/00; A61K 31/7105; A61P 11/08

(52) U.S. Cl. 424/133.1; 514/44 A; 514/1.1; 514/1.7; 514/1.8; 424/145.1; 424/158.1

(57) ABSTRACT

It is disclosed herein that blockade of inter-alpha-trypsin inhibitor (IaI) prevents the development of airway hyperresponsiveness in animal models of asthma and chronic obstructive pulmonary disease. Provided herein are methods of treating an airway disease or disorder in a subject by administering to the subject a therapeutically effective amount of an inhibitor of IaI. Also provided is a method of preventing or reducing airway hyperresponsiveness in a subject by administering to the subject a therapeutically effective amount of an inhibitor of IaI. The IaI inhibitor can be any compound that inhibits the expression or activity of IaI or a gene encoding an IaI polypeptide. In some embodiments, the IaI inhibitor is administered locally to the airway of the subject in need of treatment. For example, the inhibitor can be administered by aerosol delivery, such as by using an inhaler or nebulizer.

### CROSS REFERENCE TO RELATED APPLICATION

This application claims the benefit of U.S. Provisional Application No. 61/121,407, filed Dec. 10, 2008, which is herein incorporated by reference in its entirety.

### FIELD

This disclosure concerns the identification of inter-alpha-trypsin inhibitor (IaI) as a mediator of airway hyperresponsiveness and a therapeutic target for the treatment of human airway diseases and disorders.

### BACKGROUND

Airway disease, including asthma and chronic obstructive pulmonary disease (COPD), is a major health burden in the developed world. In 1999, asthma prevalence was reported at approximately 10,500,000 individuals in the U.S. (Mannino et al., MMWR Suffern Summ 51:1-13, 2002). The prevalence of COPD is estimated at over 23 million adults in the USA (Mannino and Braman, Proc Am Thorac Soc 4:502-506, 2007). In aggregate, airway disease affects up to 15% of the U.S. adult population and leads to a combined annual total of greater than 15,000,000 lost work days, greater than 1,100,000 hospitalizations, and more than 120,000 deaths, at an estimated cost burden of over $23 billion. A major component of airway disease is airway hyperresponsiveness (AHR), defined as the exaggerated airway constrictive response to external triggers. AHR manifests clinically as wheezing, dyspnea and cough. Since there are also asymptomatic individuals who exhibit AHR in the laboratory setting, the prevalence of AHR exceeds that of airway disease, and has been estimated at 4-35% of the general population (Jansen et al., Respir Med 91:121-134, 1997).

Currently, AHR treatment in airway disease is non-specific and consists of bronchodilators (adrenergic or anticholinergic) and immunosuppressants (corticosteroids). However, these treatments are fraught with significant side effects. Beta-agonist use has been linked to increased mortality from asthma in several studies, summarized in a meta-analysis (Salpeter et al., Ann Intern Med 144:904-912, 2006). Anticholinergic use in COPD has recently been associated with increased mortality from cardiovascular causes in these patients (Singh et al., Jama 300:1439-1450, 200). Finally, corticosteroids have a number of adverse effects, even when used topically as inhalants (Dahl, Respir Med 100:1307-1317, 2006). A major reason for the side effect profile of currently existing AHR treatments is their lack of specificity and their broad, non-targeted mechanism of action. A specific, causative and physiologic treatment of AHR would therefore greatly benefit management of airway disease patients.

The AHR response is often triggered by environmental exposures such as ozone inhalation. Ozone is a commonly encountered urban air pollutant that significantly contributes to increased morbidity (Dockery et al., N Engl J Med 329(24):1754-1759, 1993; Bell et al., JAMA 292(19):2372-2378, 2004; Gryparis et al., Am J Respir Crit Care Med 28:28, 2004; Katsouyanni et al., Eur Respir J 8(6):1030-1038, 1995) and can lead to a significant economic burden. It has been estimated that each year inhalation of ambient ozone contributes to 800 premature deaths, 4500 hospital admissions, 900,000 school absences, and more than one million restricted activity days with an estimated five billion dollar annual economic burden (Hubbell et al., Environ Health Perspect 113(1):73-82, 2005). Population-based studies suggest that for each 10-part per billion (ppb) increase in one-hour daily maximum level of ozone there is an increase in mortality risk of 0.39%-0.87%, especially in individuals with pre-existing respiratory disease (Bell et al., JAMA 292(19):2372-2378, 2004; Gryparis et al., Am J Respir Crit Care Med 28:28, 2004; Ito et al., Epidemiology 16(4):446-457, 2005; Levy et al., Epidemiology 16(4):458-468, 2005). However, the biological mechanisms that regulate the response to ambient ozone exposure remain poorly understood.

Hyaluronan is an abundant extracellular matrix component that has been recently shown to play a significant role in the response to non-infectious lung injury. Short-fragment hyaluronan (sHA) is released in the lung after sterile injury such as bleomycin instillation (Teder et al., Science 296(5565):155-158, 2002) or high-tidal-volume ventilation (Bai et al., Am J Respir Crit Care Med 172(1):92-98, 2005), and can modify the tissue response to injury. Furthermore, hyaluronan has been identified in airway secretions from asthmatics (Sahu and Lynn, Biochem J 173(2):565-568, 1978) and high molecular weight hyaluronan can attenuate the bronchoconstrictive response in exercise-induced asthma (Petrigni and Allegra, Pulm Pharmacol Ther 19(3):166-171, 2006). Given these findings, it is possible that hyaluronan may contribute to the biological response to airway injury after exposure to ozone. Thus, a need exists to indentify inhibitors of hyaluronan and hyaluronan-mediated AHR.

### SUMMARY

Disclosed herein is the finding that hyaluronan mediates ozone-induced AHR, and animals deficient in either CD44 or inter-alpha-trypsin inhibitor (IaI) are protected from ozone-induced AHR. In addition, functional blockade of IaI significantly inhibited the development of AHR in animal models of airway disease. Further disclosed is the finding that IaI levels are significantly increased in human asthmatic subjects and IaI contributes to the development of AHR in human asthma. Thus, provided herein is a method of treating an airway disease or disorder in a subject by selecting a subject in need of treatment and administering to the subject a therapeutically effective amount of an inhibitor of IaI. The IaI inhibitor is any compound that inhibits the expression or activity of IaI or a gene encoding an IaI subunit. In some embodiments, the inhibitor is an antibody, polypeptide, carbohydrate, small molecule or antisense compound.

Also provided is a method of preventing or reducing AHR in a subject, comprising administering to the subject a therapeutically effective amount of an inhibitor of IaI. In some examples, AHR is triggered by an environmental trigger, a chemical trigger, exertion or stress.

In some embodiments, the IaI inhibitor is administered locally to the airway of the subject in need of treatment. For example, the inhibitor can be administered by aerosol delivery, such as with an inhaler or nebulizer. IaI inhibitors can be administered for therapeutic or prophylactic treatment.

The foregoing and other objects, features, and advantages of the invention will become more apparent from the following detailed description, which proceeds with reference to the accompanying figures.

### BRIEF DESCRIPTION OF THE FIGURES

FIG. 1A is a graph showing total lung lavage protein 24 hours after exposure of wild-type mice (C57BL/6J), IaI-deficient mice (IaI −/−) or CD44-deficient mice (CD44 −/−) to either air or ozone. FIG. 1B is a graph showing lung lavage fluid hyaluronan level in each mouse strain 24 hours after either air or ozone exposure. FIG. 1C is an image of an agar gel following electrophoresis of concentrated lung lavage fluid hyaluronan and staining with Stains-All (Sigma, St. Louis, Mo.). Lane 1, high molecular weight (MW) hyaluronan ladder. Lane 2, low MW hyaluronan ladder. Lane 3, high MW hyaluronan (Healon). Lane 4, sonicated Healon. Lane 5, C57BL/6, ozone exposed. Lane 6, wild-type ozone exposed. Lane 7, IaI-deficient, ozone exposed. Lane 8, CD44-deficient, ozone exposed. Lane 9, hyaluronan synthase 2 (HAS2) transgenic, ozone exposed. Lane 10, representative free air exposed lavage for all strains (*p<0.001, air vs. ozone, #p<0.01 compared to C57BL/6/ozone, **p<0.05, air vs. ozone).

FIG. 2 is a series of images showing expression and localization of hyaluronan (left panels) and CD44 (middle panels), and DAPI staining (right panels), in naïve and ozone-exposed mouse lungs. Shown are immunohistochemistry results of (A) C57BL/6 mice exposed to air; (B) CD44−/− mice exposed to air; (C) C57BL/6 mice exposed to ozone; and (D) CD44−/− mice exposed to ozone. Hyaluronan is faintly visible in the subepithelial space in air-exposed mice (small arrows), but more visible after ozone exposure (big arrows). CD44 is localized in bronchial epithelial cells and macrophages (arrowheads). 400× magnification.

FIG. 3 shows a higher magnification merged image of hyaluronan and CD44 staining. Hyaluronan is found adjacent to the basal membrane below bronchial epithelia as well as surrounding subepithelial myocytes (small arrows). 600× magnification.

FIG. 4 is a series of images showing immunohistochemical staining of macrophages from C57BL/6J mice exposed to either air or ozone to evaluate cellular distribution of CD44 and hyaluronan. (A) After free air exposure, alveolar macrophages (arrowheads) stain positive for CD44 (upper left panel), but not for hyaluronan (upper right panel). (B) After ozone exposure, hyaluronan (upper left panel) and CD44 (upper right panel) co-localize on alveolar macrophages (arrows), as shown in the merged image (lower right panel). 600× magnification.

FIG. 5 includes two graphs showing the effect of methacholine treatment on total lung resistance in C57BL/6, CD44 −/− and IaI −/− mice. (A) CD44 and IaI are essential for the development of ozone-induced AHR (*p<0.01 compared to CD44- and IaI-deficient). (B) IaI but not urinary trypsin inhibitor (UTI)/bikunin injection reconstitutes AHR in IaI deficient mice (*p<0.05 compared to other groups).

FIG. 6 is a series of graphs showing treatment of C56BL/6 mice with vehicle, scrambled binding peptide (SBP), or hyaluronan-binding peptide (HABP). (A) Treatment with vehicle, SBP, or HABP does not alter ozone-induced increases in total protein in the lung lavage. (B) Treatment with vehicle, SBP, or HABP does not affect AHR in air exposed animals. (C) Hyaluronan binding protein, but not scrambled protein, significantly decreases AHR after ozone exposure (HABP vs. other groups *P<0.05).

FIG. 7 includes two graphs showing the result of ozone treatment on transgenic mice that overexpress hyaluronan synthase 2 (HAS2). (A) HAS2 Tg-positive mice, which over-express HAS2 in airway epithelia, have similar levels of total protein in lung lavage after exposure to ozone. (B) HAS2 Tg-positive animals are no different than littermate controls after exposure to filtered air, but have enhanced AHR response after exposure to ozone (*p<0.01 compared to all other groups).

FIG. 8 includes three graphs showing the effect of methacholine treatment on total lung resistance following administration of hyaluronan. (A) short-fragment hyaluronan (sHA) but not high molecular weight hyaluronan (HMW-HA) or vehicle induces AHR in naïve C57BL/6 mice (*p<0.05 compared to other groups, #p<0.05 compared to HMWHA). (B) CD44-deficient mice are resistant to sHA-induced AHR compared to C57BL/6 mice (*p<0.05, sHA treated C57BL/6 vs. sHA treated CD44−/−). (C) Instillation of HMW-HA but not vehicle before and after ozone exposure to ozone significantly ameliorates AHR (*p<0.01 vehicle vs. HMWHA, #p<0.05, vehicle vs. HMWHA).

FIG. 9 includes three graphs showing the number of cells in lung lavage from IaI-deficient and CD44-deficient mice following exposure to ozone. (A) 24 hours after ozone exposure, Id (vertical stripes) and CD44 (horizontal stripes) deficiency leads to significantly decreased numbers of inflammatory cells in the lung lavage fluid, which are mostly macrophages (white portion of bar), with few neutrophils (grey portion of bar). Injection of IaI-deficient mice with IaI (bold cross-stripes) but not equimolar bikunin (fine cross-stripes) reconstitutes the C57BL/6 phenotype (*p<0.001 compared to C57BL/6 and IaI-deficient+IaI, Bonferroni multiple comparisons testing). (B) Instillation of HABP (right hatched) but not scrambled SBP (left hatched) reduces lavage cells, which are mostly macrophages (clear portion of bar) with a few neutrophils (grey portion of bar) (*p<0.01 compared to saline and SBP treated). (C) HAS2 transgene-positive animals (hatched) have decreased lung lavage cells after ozone exposure compared to transgene-negative littermates. The difference is due to macrophages (white portion of bar) (*p<0.05 compared to HAS2 transgenic ozone-exposed).

FIG. 10 is a graph showing the effect of IaI antibody treatment on total lung resistance in mice exposed to ozone (O₃) and challenged with increasing doses of methacholine. Mice were either administered a control antibody (IgG control), monoclonal IaI antibody or polyclonal IaI antibody 24 hours after ozone exposure and then phenotyped

FIG. 11 is a graph showing the effect of IaI antibody treatment on total lung resistance in mice exposed to lipopolysaccharide (LPS) and challenged with increasing doses of methacholine. Mice were either administered a control antibody (IgG control), monoclonal IaI antibody or polyclonal IaI antibody 4 hours after LPS exposure and then phenotyped.

FIG. 12 is a graph showing the effect of IaI antibody treatment on total lung resistance in mice exposed to ovalbumin (OVA) and challenged with increasing doses of methacholine. Mice were either administered a control antibody (IgG control), monoclonal IaI antibody or polyclonal IaI antibody 48 hours after OVA exposure and then phenotyped.

FIG. 13 is a graph showing the effect of IaI deficiency on total lung resistance in mice exposed to LPS and challenged with increasing doses of methacholine (Mch). Wild-type (IaI-sufficient) and IaI-deficient mice were either unexposed or exposed to LPS.

FIG. 14 is a graph showing the effect of IaI antibody on IaI-hyaluronan binding in a competition ELISA. Plates were coated with hyaluronan and incubated with IaI and the indicated concentrations of anti-IaI antibody or IgG control. A significant decrease of IaI-hyaluronan binding is observed in the presence of anti-IaI antibody, but not in the presence of control antibody.

FIG. 15 is a graph showing the effect of IaI antibody on IaI heavy chain-hyaluronan complexing. Heavy chain transfer from IaI in plasma (1:1000) to hyaluronan was measured by ELISA. Anti-IaI antibody was used at a final dilution of 1:1000. Diluted plasma was incubated with the antibody overnight at 4° C. and the heavy chain transfer was performed thereafter by addition of TSG-6 and transfer into hyaluronan plates (*P<0.001 compared to IaI-antibody and controls).

FIG. 16 is a digital image of a western blot showing the effect of pre-incubation of plasma with monoclonal anti-IaI antibody on the transfer of heavy chains from IaI to TSG-6. Plasma was incubated with TSG-6 with or without antibody, and IaI-TSG-6 complexes were detected using appropriate antibodies in a western blot. Lane 4 (antibody lane) has a significantly less dense TSG-6-HC2 band, and there is significantly more free IaI (see left panel), indicating that IaI is not being consumed in the binding reaction.

FIG. 17 is a graph showing densitometry of N-WASP-Arp2/3 co-immunoprecipitation. Mice were exposed to inhaled endotoxin, treated with monoclonal anti-IaI antibody or IgG control and sacrificed. Tracheas were removed and homogenized, N-WASP was immunoprecipitated and the precipitate was blotted for Arp2/3. Anti-IaI-treated mice showed significantly less Arp2/3, indicating decreased N-WASP-Arp2/3 complex formation (*P<0.01).

FIG. 18A is a set of graphs showing IaI levels in bronchoalveolar lavage fluid (BALF) from atopic-asthmatic individuals and nonatopic-nonasthmatic individuals after endotoxin (LPS) segmental bronchial exposure. A significant elevation of IaI levels was detected in atopic-asthmatic individuals, but not nonatopic-nonasthmatic individuals. FIG. 18B is a set of graphs showing Id levels in BALF from atopic-asthmatic individuals and nonatopic-nonasthmatic individuals after house dust mite extract (HDM) segmental bronchial exposure. A significant elevation of IaI levels was detected in atopic-asthmatic individuals, and there is a significant decrease of IaI levels in nonatopic-nonasthmatic individuals (paired Student's t-test analysis).

FIG. 19 is a graph showing induction of AHR in naïve mice after instillation of human BALF from segmental challenge subjects. Mice received intratracheal human BALF with either anti-IaI antibody, control IgG, or vehicle (saline). Control mice received BALF from sham-exposed bronchi, or normal saline. AHR occurred in mice receiving BALF and saline or IgG, whereas mice receiving BALF and IaI antibody were comparable to controls (*P<0.001, **P<0.05, ANOVA with Bonferroni post-hoc analysis).

### DETAILED DESCRIPTION

### I. Introduction

AHR is defined as the exaggerated response of the airways to internal or external stimuli, and is a major component of airway diseases such as asthma or COPD. AHR is often triggered by environmental exposures to pollutants such as ozone. Ozone is a common urban environmental air pollutant and significantly contributes to hospitalizations for respiratory illness. The mechanisms that regulate ozone-induced bronchoconstriction remain poorly understood. Hyaluronan was recently shown to play a central role in the response to non-infectious lung injury.

Applicants set out to test the hypothesis that hyaluronan may contribute to airway hyperreactivity after exposure to ambient ozone. As disclosed herein, the role of hyaluronan in airway hyperresponsiveness (AHR) was evaluated using an established model of ozone-induced airways disease. The role of hyaluronan in response to ozone was determined using therapeutic blockade, genetically modified animals, and direct challenge to hyaluronan. Ozone-exposed mice exhibit enhanced AHR associated with elevated hyaluronan levels in the lavage fluid. Mice deficient in either CD44 (the major receptor for hyaluronan) or inter-alpha-trypsin inhibitor (a molecule which facilitates hyaluronan binding) show similar elevations in hyaluronan, but are protected from ozone-induced AHR. The findings described herein demonstrate that hyaluronan mediates ozone-induced AHR, which is dependent on both CD44 and inter-alpha-trypsin inhibitor (IaI).

It is further shown herein that functional blockade of IaI, using either polyclonal or monoclonal antibodies specific for IaI, significantly diminishes airway hyperreactivity in three different animals models of airway disease. Also described herein is the finding that human asthmatic subjects have significantly increased IaI levels in bronchoalveolar lavage fluid (BALF), compared to their baseline levels, after exposure to lipopolysaccharide (LPS) or house dust mite (HDM) antigen, which is in contrast to non-asthmatic individuals who have no appreciable change in IaI levels. Administration of BALF from the LPS and HDM-exposed asthmatic patients to naïve mice induces AHR in the mice, an effect that is prevented by co-administration of an IaI-specific antibody.

Based on these findings, inhibitors of IaI are contemplated for use in the treatment of a number of different human airway diseases, including asthma, chronic obstructive pulmonary disease (COPD), cystic fibrosis and acute or chronic bronchiolitis.

### II. Abbreviations

AHR Airway hyperresponsiveness

AMBP α-1-microglobulin/bikunin precursor

BALF Bronchoalveolar lavage fluid

BOOP Bronchiolitis obliterans organizing pneumonia

CF Cystic fibrosis

COPD Chronic obstructive pulmonary disease

DPB Diffuse panbronchiolitis

DPI Dry powder inhaler

ELISA Enzyme-linked immunosorbent assay

FA Filtered air

HA Hyaluronan

HABP Hyaluronan binding protein

HAS2 Hyaluronan synthase 2

HDM House dust mite

HMW-HA High molecular weight-hyaluronan

IaI Inter-alpha trypsin inhibitor

i.p. Intraperitoneal

ITI Inter-alpha (globulin) inhibitor or inter-alpha trypsin inhibitor

ITIH ITI heavy chain

i.v. Intravenous

LMWH Low molecular weight heparin

LPS Lipopolysaccharide

MDI Metered dose inhaler

miRNA MicroRNA

MW Molecular weight

O₃ Ozone

OVA Ovalbumin

PBS Phosphate-buffered saline

ppb Parts per billion

ppm Parts per million

psi Pounds per square inch

RNA Ribonucleic acid

RT-PCR Reverse transcriptase polymerase chain reaction

SBP Scrambled binding peptide

sHA Short-fragment hyaluronan

s.c. Subcutaneous

shRNA Short hairpin RNA

siRNA Small interfering RNA

Tg Transgenic

ULMWH Ultra low molecular weight heparin

UTI Urinary trypsin inhibitor

UV Ultraviolet

### III. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Acute: As used herein, an “acute” disease or disorder refers to a disease or disorder of short duration, generally characterized by severe symptoms and rapid progression. This term is used in contrast to “chronic”.

Administration: Administration of an active compound or composition (such as a compound comprising an IaI inhibitor), which can occur by any route known to one of skill in the art. Administration can be local or systemic. Examples of local administration (also referred to as “local delivery”) include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration (for example, by aerosol delivery). In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Aerosol: A gaseous suspension of fine solid or liquid particles, such as a suspension of a drug or other substance to be dispensed in a cloud or mist. Aerosol delivery refers to administration (such as to the airway) of a therapeutic agent that is formulated as an aerosol.

Agent: Any substance or any combination of substances that is useful for achieving an end or result; for example, a substance or combination of substances useful for modulating gene expression or protein activity, or inhibiting AHR. In some embodiments, the agent is a therapeutic agent, such as a therapeutic agent for the treatment of an airway disease or disorder.

Airway disease or disorder: Includes any disease or disorder that effects the respiratory tract (such as the lungs, mouth, nose, pulmonary alveoli, pharynx, larynx, trachea, and bronchi). In many cases, airway diseases or disorders result in airway constriction with symptoms including wheezing, coughing and shortness of breath. In some embodiments herein, the airway disease or disorder is a chronic disorder, such as, but not limited to, asthma, chronic obstructive pulmonary disease, cystic fibrosis, obliterative bronchiolitis, diffuse panbronchiolitis or cryptogenic organizing pneumonia. In other embodiments, the airway disease or disorder is an acute disease or disorder, such as, but not limited to exercise-induced asthma, airway hyperresponsiveness, respiratory infection, acute bronchiolitis, pollution-induced airway injury, chemical-induced airway injury and ventilation-induced airway injury.

Airway hyperresponsiveness (AHR): Refers to a state that is characterized by increased susceptibility to airway narrowing (also referred to as bronchospasm, the contraction of the bronchioles or small airways), following exposure to a trigger, such as an environmental trigger (e.g., pollution or an allergen). Hyperreactivity can be assessed using constrictor agonists, such as methacholine or histamine. Subjects with AHR have a lower threshold of tolerance to constrictor agonists compared to healthy subjects. AHR is a hallmark of asthma, but also occurs in many other airway diseases such as COPD. AHR is also known as bronchial hyperresponsiveness or airway hyperreactivity.

Airway injury: Refers to any type of physical or structural damage to the airway, such as from trauma (for example, an injury to the airway resulting from intubation/ventilation) or exposure to a chemical (such as a chemical burn from ammonia or a toxic gas).

Allergen: Any substance that can produce an allergic reaction or hypersensitivity in a subject. For example, common allergens include pollen, dander, mold, drugs (such as antibiotics) or particular types of food (for example, eggs).

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. As used herein, the term antibody includes intact immunoglobulins as well as a number of well-characterized fragments produced by digestion with various peptidases, or genetically engineered artificial antibodies. Antibodies for use in the methods and compositions of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988).

Also specifically contemplated are human antibodies (arising from human genes) and humanized antibodies, either of which is suitable for administration to humans without engendering an adverse immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following methods known in the art, such as by substituting rodent CDRs or CDR sequences for the corresponding sequences of a human antibody (see, for instance, U.S. Pat. No. 5,225,539; Jones et al., Nature 321(6069):522-525, 1986; Riechmann et al., J Mol Biol. 203(3):825-828, 1988; Verhoeyen et al., Science 239(4847):1534-1536, 1988; Riechmann et al., Nature 332(6162):323-327 1988; or Verhoeyen et al., Bioessays 8(2):74-78, 1988). Antibodies specific for IaI are known in the art (see, for example, U.S. Pat. No. 6,660,482; U.S. Patent Application Publication No. 2007/0297982; and Lim et al., J. Infect. Dis. 188:919-926, 2003).

Antisense compound: Refers to an oligomeric compound that is at least partially complementary to the region of a target nucleic acid molecule to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. In one embodiment, the target nucleic acid molecule is a nucleic acid molecule encoding an IaI polypeptide.

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound.

Antisense oligonucleotide: As used herein, an “antisense oligonucleotide” is a single-stranded antisense compound that is a nucleic acid-based oligomer. An antisense oligonucleotide can include one or more chemical modifications to the sugar, base, and/or internucleoside linkages. Generally, antisense oligonucleotides are “DNA-like” such that when the antisense oligonucleotide hybridizes to a target mRNA, the duplex is recognized by RNase H (an enzyme that recognizes DNA:RNA duplexes), resulting in cleavage of the mRNA.

Binding affinity: A term that refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

Asthma: A chronic condition involving the respiratory system in which the airways constrict, become inflamed and are lined with excessive amounts of mucus, often in response to one or more triggers. Episodes of asthma can be triggered by a number of different factors, such as exposure to an environmental stimulant, such as an allergen, environmental tobacco smoke, cold or warm air, perfume, pet dander, moist air, exercise or exertion, or emotional stress. In children, the most common triggers are viral illnesses such as those that cause the common cold. The airway narrowing that occurs in asthma causes symptoms such as wheezing, shortness of breath, chest tightness and coughing.

Bronchiolitis: Inflammation of the bronchioles, the smallest air passages of the lungs. The term often refers to acute viral bronchiolitis, a common disease in infancy, usually caused by respiratory syncytial virus or other viruses including metapneumovirus, influenza, parainfluenza, coronavirus, adenovirus and rhinovirus. Obliterative bronchiolitis (also known as bronchiolitis obliterans or constrictive bronchiolitis) is a life-threatening form of non-reversible obstructive lung disease in which the bronchioles are plugged with granulation tissue. Inflammation and scarring occur in the airways of the lung, resulting in severe shortness of breath and dry cough. Obliterative bronchiolitis has many possible causes, including collagen vascular disease, transplant rejection in organ transplant patients, viral infection (e.g., respiratory syncytial virus, adenovirus, human immunodeficiency virus or cytomegalovirus), pneumocystis pneumonia, drug reaction, complications of prematurity (bronchopulmonary dysplasia), and exposure to toxic fumes (such as diacetyl, sulfur dioxide, nitrogen dioxide, ammonia, chlorine, thionyl chloride, methyl isocyanate, hydrogen fluoride, hydrogen bromide, hydrogen chloride, hydrogen sulfide, phosgene, polyamide-amine dyes or ozone). Diffuse panbronchiolitis (DPB) is an inflammatory lung disease (considered to be a type of COPD) with no known cause. DPB is a severe, progressive form of bronchiolitis, mainly affecting the respiratory bronchioles (the section of the bronchioles involved in gas exchange). If left untreated, DPB is fatal, usually progressing to bronchiectasis, an irreversible lung condition that causes respiratory failure.

Bronchiolitis obliterans organizing pneumonia (BOOP): An inflammation of the bronchioles and surrounding tissue in the lungs. BOOP is often caused by a pre-existing chronic inflammatory disease, such as rheumatoid arthritis. BOOP can also be a side effect of certain medicinal drugs (e.g. amiodarone). In cases where no cause is found, the disease is referred to as cryptogenic organizing pneumonia. The clinical features and radiological imaging resemble infectious pneumonia. However, diagnosis is suspected after there is no response to multiple antibiotics, and blood and sputum cultures are negative for organisms. “Organizing” refers to unresolved pneumonia (in which the alveolar exudate persists and eventually undergoes fibrosis) in which fibrous tissue forms in the alveoli. The phase of resolution and/or remodeling following bacterial infections is commonly referred to as organizing pneumonia, both clinically and pathologically.

Carbohydrate: An organic compound made up of carbon, hydrogen and oxygen atoms. Carbohydrates are a large group of compounds that include sugars, starch and cellulose. In particular examples herein, the carbohydrate is a heparin molecule.

Chronic: A “chronic” disease or disorder is a condition that persists for a long period of time. Any disease or disorder that persists for at least three months is generally considered a “chronic” disease or disorder.

Chronic obstructive pulmonary disease (COPD): A disease of the lungs in which the airways become narrowed, leading to a limitation of the flow of air to and from the lungs, which causes shortness of breath. In contrast to asthma, the limitation of airflow is poorly reversible and usually gradually gets worse over time. COPD is caused by noxious particles or gases, most commonly from smoking, which trigger an abnormal inflammatory response in the lung. The inflammatory response in the larger airways is known as chronic bronchitis, which is diagnosed clinically when people regularly cough up sputum. In the alveoli, the inflammatory response causes destruction of the tissue of the lung, a process known as emphysema. The natural course of COPD is characterized by occasional sudden worsening of symptoms called acute exacerbations, most of which are caused by infections or air pollution. COPD is also known as chronic obstructive lung disease, chronic obstructive airway disease, chronic airflow limitation and chronic obstructive respiratory disease. As an example, emphysema is one type of COPD.

Cystic fibrosis (CF): A hereditary (autosomal recessive) disease affecting the exocrine (mucus) glands of the lungs, liver, pancreas, and intestines, causing progressive disability due to multisystem failure. CF is caused by a mutation in a gene called the cystic fibrosis transmembrane conductance regulator. The product of this gene is a chloride ion channel important in creating sweat, digestive juices, and mucus. Thick mucus production in CF patients results in frequent lung infections. Lung disease results from clogging the airways due to mucosa buildup and resulting inflammation. Inflammation and infection cause injury to the lungs and structural changes that lead to a variety of symptoms. In the early stages, incessant coughing, copious phlegm production and decreased ability to exercise are common Many of these symptoms occur when bacteria that normally inhabit the thick mucus grow out of control and cause pneumonia. In later stages of CF, changes in the architecture of the lung further exacerbate chronic difficulties in breathing.

Glycosaminoglycan (GAG): Polysaccharide composed of disaccharide subunits of N-acetyl-hexosamine and hexose or hexuronic acid, with varying degrees of sulfation occurring on each subunit. GAGs include heparin, heparin sulfate, chondroitin sulfate, dermatan sulfate, and heparan sulfate. In some embodiments of the disclosure, the GAG is chondroitin sulfate, a heparin sulfate or a heparin molecule.

Heparin: A highly sulfated glycosaminoglycan (also referred to as a mucopolysaccharide) released by mast cells and basophils in many tissues, particularly the liver and lungs. Heparin is known to have potent anticoagulant properties. Heparin and other members of the glycosaminoglycan family play significant roles in a diverse set of biological processes, including blood coagulation, virus infection, cell growth, inflammation, wound healing, tumor metastasis, lipid metabolism, and diseases of the nervous system. Unfractionated heparin is a variable mixture of saccharide polymers with molecular weights ranging from approximately 5,000-30.000 daltons. Low molecular weight heparins (LMWH) are obtained by the chemical or enzymatic depolymerization of heparin giving rise to mixtures of smaller polymers with weight ranges from 2,000-15,000 daltons. Ultra low molecular weight heparins (ULMWH) are generally classified as heparins with a molecular weight of less than 3,000 daltons. A number of heparins are commercially available and marketed for clinical use, including preparations of unfractionated heparin, LMWH (including tinzaparin, dalteparin and enoxaparin) and fondaparinux, a synthetic pentasaccharide. Heparin, heparin derivatives and heparin-like molecules are known in the art (see, for example, U.S. Patent Application Publication Nos. 2008/0171722 and 2008/0014239).

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 60% identity)

Hybridization: 6×SSC at RT to 55° C. for 16-20 hours

Wash at least twice: 2×-3×SSC at RT to 55° C. for 20-30 minutes each.

Inhaler: An apparatus for administering vapor or volatilized medications by inhalation. Inhalers are often used to administer medication locally to the airway, for example to treat asthma. In some examples, the inhaler is a dry powder inhaler. In other examples, the inhaler is a metered-dose inhaler.

Inhibitor of inter-alpha-trypsin inhibitor (IaI): As used herein, an “inhibitor” of inter-alpha-trypsin inhibitor (IaI) is any compound that inhibits the expression or activity of IaI, or inhibits expression of a gene encoding an IaI subunit, such as the gene encoding the IaI light chain (bikunin) or one of the five IaI heavy chains (ITIH1, ITIH2, ITIH3, ITIH4, ITIH5).

In some embodiments, the inhibitor is an antibody, polypeptide, carbohydrate, small molecule or antisense compound. An inhibitor of the disclosure, for example, can inhibit the activity of IaI either directly or indirectly. Direct inhibition can be accomplished, for example, by binding to IaI and thereby preventing the protein from binding an intended target. Indirect inhibition can be accomplished, for example, by binding to a protein's intended target, such as a receptor or binding partner, thereby blocking or reducing activity of the protein. Furthermore, an inhibitor of the disclosure can inhibit a gene encoding an IaI subunit by reducing or inhibiting expression of the gene, inter glia by interfering with gene expression (transcription, processing, translation, post-translational modification), for example, by interfering with the gene's mRNA and blocking translation of the gene product or by post-translational modification of a gene product, or by causing changes in intracellular localization.

Inter-alpha-trypsin inhibitor (IaI): A molecule consisting of a light chain (L) serine protease inhibitor, known as bikunin or urinary trypsin inhibitor (UTI), and one or two heavy chains (H) containing von-Willebrand type A (vWA) domains. There are five known IaI heavy chain polypeptides, which are encoded by inter-alpha (globulin) inhibitor (ITI) H1, ITIH2, ITIH3, ITIH4 and ITIH5. Bikunin is encoded by α-1-microglobulinibikunin precursor (AMBP). IaI is assembled in the liver and released into the circulation, and can be found in fairly high concentrations in mammalian serum. While IaI is most abundantly found in the liver, IaI is also present in the lungs. The IaI heavy chains, also known as serum-derived hyaluronan-associated proteins, mediate binding of IaI to hyaluronic acid in the extracellular matrix. IaI is also known as inter-alpha (globulin) inhibitor.

MicroRNA (miRNA): Single-stranded RNA molecules that regulate gene expression. miRNAs are generally 21-23 nucleotides in length. miRNAs are processed from primary transcripts known as pri-miRNA to short stem-loop structures called pre-miRNA and finally to functional miRNA. Mature miRNA molecules are partially complementary to one or more messenger RNA molecules, and their primary function is to down-regulate gene expression. MicroRNAs regulate gene expression through the RNAi pathway.

Nebulizer: A device that turns liquid forms of medicine into a fine spray (aerosol) that can be inhaled, especially for delivering medication to the deep part of the respiratory tract.

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Patient: As used herein, the term “patient” includes human and non-human animals such as companion animals (dogs and cats and the like) and livestock animals that have been diagnosed with a disease or disorder and/or are in need of therapeutic treatment. The preferred patient for treatment is a human.

Percent identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Res. 16:10881-10890, 1988; Pearson and Lipman, Proc. Natl. Acad. Sci. USA 85:2444, 1988; and Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some embodiments, the pharmaceutically acceptable carrier is suitable for delivery to an airway. Carriers for airway delivery are well known in the art and are discussed below.

Polypeptide: A polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

The term polypeptide fragment refers to a portion of a polypeptide that exhibits at least one useful epitope. The phrase “functional fragment(s) of a polypeptide” refers to all fragments of a polypeptide that retain an activity, or a measurable portion of an activity, of the polypeptide from which the fragment is derived. Fragments, for example, can vary in size from a polypeptide fragment as small as an epitope capable of binding an antibody molecule to a large polypeptide capable of participating in the characteristic induction or programming of phenotypic changes within a cell. An epitope is a region of a polypeptide capable of binding an immunoglobulin generated in response to contact with an antigen.

Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

In some circumstances, variations in the cDNA sequence that result in amino acid changes, whether conservative or not, are minimized in order to preserve the functional and immunologic identity of the encoded protein. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80%, 90%, or even 95% or 98% identical to the native amino acid sequence. Programs and algorithms for determining percentage identity can be found at the NCBI website.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Ribozyme: A catalytic RNA molecule. In some cases, ribozymes can bind to specific sites on other RNA molecules and catalyze the hydrolysis of phosphodiester bonds in the RNA molecules.

RNA interference (RNAi): Refers to a cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. Double-stranded RNA molecules that inhibit gene expression through the RNAi pathway include siRNAs, miRNAs, and shRNAs.

Short hairpin RNA (shRNA): A sequence of RNA that makes a tight hairpin turn and can be used to silence gene expression via the RNAi pathway. The shRNA hairpin structure is cleaved by the cellular machinery into siRNA.

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule inhibitor: A molecule, typically with a molecular weight less than about 1000 Daltons, or in some embodiments, less than about 500 Daltons, wherein the molecule is capable of inhibiting, to some measurable extent, an activity of some target molecule. In some embodiments, the target molecule is IaI.

Subject: Living multi-cellular vertebrate organisms, a category that includes both human and non-human mammals.

Therapeutic: A generic term that includes both diagnosis and treatment.

Therapeutically effective amount: A quantity of a specified pharmaceutical agent sufficient to achieve a desired effect in a subject, or in a cell, being treated with the pharmaceutical agent. For example, this can be the amount of an antibody, polypeptide, carbohydrate, small molecule or antisense compound useful for inhibiting expression or activity of IaI. The effective amount of the pharmaceutical agent will be dependent on several factors, including, but not limited to the subject or cells being treated, and the manner of administration of the therapeutic composition.

Trigger: As used herein, a “trigger” for AHR is any type of environmental, chemical or physical element or perturbation that causes or increases the risk of AHR. In some examples, an environmental trigger is pollution (such as ozone or particulate matter) or an allergen. In some examples, a chemical trigger is exposure to ammonia or another toxic gas. In some examples, the trigger is stress or exertion.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### IV. Overview of Several Embodiments

Described herein is the finding that inhibition of IaI prevents airway hyperresponsiveness (AHR) in animal models of airway disease. Further disclosed is the finding that IaI levels are significantly increased in human asthmatic subjects and IaI contributes to the development of AHR in human asthma. Thus, provided herein is a method of treating an airway disease or disorder in a subject by selecting a subject in need of treatment and administering to the subject a therapeutically effective amount of an inhibitor of IaI, thereby treating the airway disease or disorder.

In some embodiments, the airway disease or disorder is a chronic disease or disorder. Chronic airway diseases and disorders include, but are not limited to, asthma, chronic obstructive pulmonary disease, cystic fibrosis, bronchiolitis (including obliterative bronchiolitis and diffuse panbronchiolitis) and cryptogenic organizing pneumonia. In other embodiments, the airway disease or disorder is an acute disease or disorder. Acute airway diseases and disorders include, but are not limited to, exercise-induced asthma, airway hyperresponsiveness, respiratory infection, acute bronchiolitis, pollution-induced airway injury, chemical-induced airway injury and ventilation-induced airway injury.

Also provided herein is a method of preventing or reducing airway hyperresponsiveness (AHR) in a subject, comprising administering to the subject a therapeutically effective amount of an inhibitor of IaI, thereby preventing or reducing AHR. In some embodiments, the subject suffers from asthma or COPD. In some embodiments, AHR is triggered by an environmental trigger (such as ozone or particulate matter), a chemical trigger (such as ammonia or another toxic chemical), exertion or stress. For the treatment of AHR, the IaI inhibitor can either be administered prophylactically prior to exposure to the trigger, or therapeutically after the onset of symptoms.

IaI inhibitors useful for the methods provided herein are any compound(s) that inhibits the expression or activity of IaI or a gene encoding an IaI subunit. In some embodiments, the inhibitor is an antibody, polypeptide, carbohydrate, small molecule or antisense compound. When the IaI inhibitor is an antibody, the antibody can be a polyclonal antibody or a monoclonal antibody. In particular examples, the antibody is a humanized antibody, a chimeric antibody (such as an antibody having both human and mouse sequences) or a fully human antibody. In some cases, the antibody is a single-chain antibody (scFv). In some examples, the IaI inhibitor is an organic molecule, such as a carbohydrate, for example a heparin molecule. Heparin molecules include heparin derivatives, and both high and low molecular weight natural and synthetic heparins. In particular examples, the heparin molecule is a low molecular weight heparin (LMWH) or an ultra low molecular weight heparin (ULMWH).

In some embodiments, the IaI inhibitor is an antisense compound. Antisense compounds include, but are not limited to antisense oligonucleotides, siRNAs, miRNAs, shRNAs or ribozymes. The antisense compound is targeted to one or more genes encoding an IaI subunit. In some examples, the antisense compound specifically hybridizes with the gene encoding the IaI light chain (AMBP). In other examples, the antisense compound specifically hybridizes with a gene encoding an IaI heavy chain (ITIH1, ITIH2, ITIH3, ITIH4 or ITIH5).

The IaI inhibitor can be administered to the patient using any suitable route of administration. In some embodiments, the IaI inhibitor is administered locally to the airway of the subject in need of treatment. For example, the inhibitor can be administered by aerosol delivery, such as by using an inhaler or nebulizer. Inhalers include, for example, metered dose inhalers and dry powder inhalers.

### V. Treatment of Airway Disease using IaI Inhibitors

It is disclosed herein that IaI plays an important role in the development and progression of airway hyperreactivity. Furthermore, local administration of an IaI inhibitor significantly reduces AHR in animal models of airway disease. Thus, provided herein is a method of treating an airway disease or disorder by administering to a subject in need of treatment an inhibitor of IaI. AHR is involved in a number of airway diseases and disorders. Accordingly, IaI inhibitors are contemplated for use in the treatment of a number of conditions, including both acute and chronic diseases and disorders of the airway.

In some embodiments, an IaI inhibitor is used to treat a chronic airway disease or disorder. Chronic airway diseases and disorders, include, but are not limited to asthma, COPD, chronic cough, cystic fibrosis, cryptogenic organizing pneumonia and bronchiolitis, including obliterative bronchiolitis and diffuse panbronchiolitis. In other embodiments, an IaI inhibiter is used to treat an acute airway disease or disorder. Acute airway diseases and disorders include, but are not limited to, acute asthma exacerbations, exercise-induced asthma, airway hyperresponsiveness, respiratory infection, acute bronchiolitis (infectious or noninfectious), pollution-induced airway injury, chemical-induced airway injury and ventilation-induced airway injury.

In some cases, the patient selected for treatment has a chronic disease or disorder, such as a chronic airway disease or disorder listed above. In other cases, the patient selected for treatment is a patient that has been exposed to a trigger of AHR. For example, potential triggers of AHR include pollution (such as ozone or particulate matter), chemical accidents that cause airway burns (such as exposure to ammonia or other toxic chemicals), and exertion or stress (particularly for asthma sufferers). Respiratory infection (such as viral infection), which often causes bronchiolitis, can also result in AHR. IaI inhibitors can also be used to treat patients having an airway injury, such as an injury resulting from intubation/ventilation or a chemical burn.

IaI inhibitors can be used therapeutically to treat acute or chronic airway diseases, or prophylactically. For example, prophylactic use of IaI inhibitors includes treatment of patients at risk of exercise-induced asthma, or at risk of environmental exposure to pollutants or chemicals.

Also provided herein is a method of preventing or reducing airway hyperresponsiveness (AHR) in a subject, comprising administering to the subject a therapeutically effective amount of an inhibitor of IaI, thereby preventing or educing AHR. The subject can be any subject that is suffering from AHR, or is prone to AHR. In some cases, the subject has a chronic airway disease, such as asthma or COPD, chronic bronchitis, eosinophilic bronchitis, cough-variant asthma, chronic cough, obliterative bronchiolitis, diffuse panbronchiolitis, hypersensitivity pneumonitis, or any other type of airway disease involving airway constriction.

AHR can be triggered by any one of a number of factors. In some embodiments, AHR is triggered by an environmental trigger, a chemical trigger, exertion or stress. In particular examples, the environmental trigger is pollution, such as ozone or particulate matter, or an allergen. In some embodiments, the IaI inhibitor is administered prophylactically prior to exposure to the trigger. For example, patients with asthma can be treated with an IaI inhibitor prior to exercise or prior to exposure to an environmental trigger, such as pollution or an allergen. As another example, prophylactic treatment can also be useful for treating subjects at risk for chemical exposure, such as first responders to a chemical accident.

A. Administration of IaI Inhibitors by Aerosol Delivery

In some cases, it is desirable to deliver an IaI inhibitor locally to the airway to limit potential side effects that may result from inhibiting IaI in other cells or tissues, such as the liver. Therefore, in some embodiments, the IaI inhibitor is administered to the subject in need of treatment by aerosol delivery. Aerosol delivery is generally of lower risk than systemic delivery as it allows for administration of smaller doses of the inhaled medication with equal or greater therapeutic effect and minimal adverse effects. The therapeutic efficiency of therapeutic agents (such as compositions comprising an IaI inhibitor) administered by aerosolization depends not only on the pharmacological properties of the therapeutic agents themselves, but also on the characteristics of the delivery device. The characteristics of the delivery device influence the amount of drug deposited in the lungs and the pattern of drug distribution in the airways.

Aerosols are airborne suspensions of fine particles. The particles may be solids or liquids. Aerosol particles are heterodisperse (i.e. the particles include a range of sizes) and aerosol particle size distribution is best described by a log normal distribution. Particles tend to settle (sediment), adhere to each other (coagulate), and adhere to structures such as tubing and mucosa (deposit). The particles delivered by aerosol can be conveniently characterized on the basis of their aerodynamic behavior. One parameter is the mass median aerodynamic diameter (MMAD). By definition, a particle distribution with an MMAD of 1 μM has the same average rate of settling as a droplet of unit density and 1 μM diameter.

The size of an aerosol particle, as well as variables affecting the respiratory system, influence the deposition of inhaled aerosols in the airways. For example, particles larger than 10 μM in diameter are unlikely to deposit in the lungs. However, particles smaller than 0.5 μM are likely to reach the alveoli or may be exhaled. Therefore, particles that have a diameter of between 1 μM and 5 μM are most efficiently deposited in the lower respiratory tract. Particles of these sizes are most efficient for the delivery of therapeutic agents for some airway diseases, such as asthma.

The percentage of the aerosol mass contained within respirable droplets (i.e., droplets with a diameter smaller than 5 depends on the inhalation device being used. Slow, steady inhalation increases the number of particles that penetrate the peripheral parts of the lungs. As the inhaled volume is increased, the aerosol can penetrate more peripherally into the bronchial tree. A period of breath-holding, on completion of inhalation, enables those particles that have penetrated to the lung periphery to settle into the airways via gravity. Increased inspiratory flow rates, typically observed in patients with acute asthma, result in increased losses of inhaled drug. This occurs because aerosol particles impact in the upper airway and at the bifurcations of the first few bronchial divisions. Other factors associated with pulmonary airway disease may also alter aerosol deposition. Airway obstruction and changes in the pulmonary parenchyma are often associated with pulmonary deposition in the peripheral airways in patients with asthma.

With aerosol delivery, the nose efficiently traps particles before their deposition in the lung. Therefore, mouth breathing of the aerosolized particles is preferred. The aerosolized particles are lost from many sites. Generally, the amount of the nebulized dose reaching the small airways is less than about 15%. In many cases, approximately 90% of the inhaled dose is swallowed and then absorbed from the gastrointestinal tract. The small fraction of the dose that reaches the airways is also absorbed into the blood stream. The swallowed fraction of the dose is, therefore, absorbed and metabolized in the same way as an oral formulation, while the fraction of the dose that reaches the airways is absorbed into the blood stream and metabolized in the same way as an intravenous dose.

B. Inhalation Devices

Typically, aerosol delivery is accomplished using an inhaler, such as a metered dose inhaler (MDI) or a dry powder inhaler (DPI), or a nebulizer. Inhalers and nebulizers are devices for administering aerosolized therapeutic agents to a subject via inhalation. Ultrasonic, electrical, pneumatic, hydrostatic or mechanical forces (such as compressed air or by other gases) can drive these devices.

A nebulizer delivers fine mists of liquids, suspensions or dispersions for inhalation. A nebulizer can be a mechanical powder device which disperses fine powder into a finer mist using leverage or piezo electric charges in combination with suitably manufactured porous filter discs, or as formulations that do not aggregate in the dose chamber. Propellants can be used to spray a fine mist of the product such as fluorochlorocarbons, fluorocarbons, nitrogen, carbon dioxide, or other compressed gases. Nebulized aerosols are particularly useful for children under five years of age and in the treatment of severe asthma where respiratory insufficiency may impair inhalation from an MDI or dry powder inhaler.

A nebulizer type inhalation delivery device can contain a therapeutic agent (such as an IaI inhibitor) as a solution, usually aqueous, or a suspension. In generating the nebulized spray of the therapeutic agent for inhalation, the nebulizer type delivery device can be driven ultrasonically, by compressed air, by other gases, electronically or mechanically. The ultrasonic nebulizer device generally works by imposing a rapidly oscillating waveform onto the liquid film of the formulation via an electrochemical vibrating surface. At a given amplitude, the waveform becomes unstable, disintegrates the liquids film, and produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer can be portable and hand held in design, and can be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer can use a mechanical actuator to force the liquid formulation through a multiorifice nozzle of defined aperture size to produce an aerosol of the formulation for inhalation. In the design of single dose nebulizers, blister packs containing single doses of the fomulation can be employed.

Formulations suitable for use with a nebulizer, either jet or ultrasonic, can include a compound (such as an IaI inhibitor) dissolved in water at a concentration of about 0.1 to 25 mg of biologically active compound per mL of solution. The formulation can also include a buffer and a simple sugar (such as for protein stabilization and regulation of osmotic pressure). The nebulizer formulation can also contain a surfactant, to reduce or prevent surface induced aggregation of the compound (such as a protein) caused by atomization of the solution in forming the aerosol (U.S. Patent Application Publication No. 2007/0065367).

A metered dose inhalator (MDI) can also be employed as the aerosol delivery device. Because of their convenience and effectiveness, MDIs are probably the most widely used therapeutic aerosol used for inhaled drug delivery to outpatients. MDIs are pressurized and their basic structure consists of a metering valve, an actuator and a container. A propellant is used to discharge the formulation from the device. The composition can include particles of a defined size suspended in the pressurized propellant liquid, or the composition can be in a solution or suspension of pressurized liquid propellant. The propellants used are primarily atmospheric friendly hydroflourocarbons. Traditional chloroflourocarbons, such as CFC-11, 12 and 114, are used only when essential. The device of the inhalation system can deliver a single dose (such as by a blister pack), or it can be multi-dose in design. To ensure accuracy of dosing, the delivery of the formulation can be programmed via a microprocessor to occur at a certain point in the inhalation cycle. In some cases, the MDI can be portable and hand held.

For optimal pulmonary drug deposition, the medication should be released at the beginning of a slow inspiration that lasts about five seconds and is followed by 10 seconds of breath-holding. Several inhalation aids have been designed to improve the effectiveness of MDIs. These are most useful in patients who have poor hand-to-breath coordination. A short tube (for example, cones or spheres) may be used to direct the aerosol straight into the mouth or collapsible bags can act as an aerosol reservoir holding particles in suspension for three to five seconds, during which time the patient can inhale the drug. However, when any of these devices is used, aerosol velocity upon entering the oropharynx is decreased and drug availability to the lungs and deposition in the oropharynx is decreased. Formulations for use with a MDI device generally includes a finely divided powder containing the compound (such as an IaI inhibitor) suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant (U.S. Patent Application Publication No. 2007/0065367).

A dry powder inhalator (DPI) also can be used as the aerosol delivery device. DPIs are often used to deliver agents to patients who have difficulty using a MDI (for example, children and elderly patients). The basic design of a DPI includes a metering system, a powdered composition and a method to disperse the composition. Forces like rotation and vibration can be used to disperse the composition. The metering and dispersion systems can be mechanically or electrically driven and can be microprocessor-programmable. The device can be portable and hand held. The inhalator can be multi- or single-dose in design and use such options as hard gelatin capsules or blister packages for accurate unit doses.

The therapeutic composition (such as a composition comprising an IaI inhibitor) can be dispersed from the device by passive inhalation (such as the patient's own inspiratory effort), or an active dispersion system can be employed. The dry powder of the therapeutic composition can be sized via processes such as jet milling, spray dying and supercritical fluid manufacture. Acceptable excipients such as the sugars mannitol and maltose can be used in the preparation of the powdered formulations.

Formulations for dispensing from a powder inhaler device may comprise a finely divided dry powder containing the compound (such as an IaI inhibitor) and can also include a bulking agent, such as lactose, sorbitol, sucrose, or mannitol in amounts which facilitate dispersal of the powder from the device, for example, 50 to 90% by weight of the formulation. The compound can be prepared in particulate form with an average particle size of less than 10 μM, such as 0.5 to 5 μM, for delivery to the distal lung (U.S. Patent Application Publication No. 2007/0065367).

Exemplary airway delivery methods, inhalation devices and formulations are known in the art (see, for example, U.S. Patent Application Nos. 2004/0009126 and 2007/0065367).

### VI. Antibodies Specific for IaI

An IaI polypeptide or a fragment or conservative variant thereof can be used to produce antibodies which are immunoreactive or specifically bind to an epitope of IaI. IaI is composed of a light chain, known as bikunin, and two heavy chains. There are five known heavy chains of IaI, including inter-alpha (globulin) inhibitor (ITI) H1 polypeptide, ITIH2 polypeptide, ITIH3 polypeptide, ITIH4 polypeptide and ITIH5 polypeptide. As used herein, an “antibody specific for IaI” includes antibodies that specifically bind one or more of the light chain or heavy chain polypeptides of IaI.

Antibodies contemplated for use in the methods provided herein include, but are not limited to the IaI-specific monoclonal and polyclonal antibodies described in U.S. Pat. No. 6,660,482; U.S. Patent Application Publication No. 2007/0297982; and Lim et al. (J. Infect. Dis. 188:919-926, 2003), or any fragments thereof.

Polyclonal antibodies, antibodies which consist essentially of pooled monoclonal antibodies with different epitopic specificities, as well as distinct monoclonal antibody preparations are included. The preparation of polyclonal antibodies is well known to those skilled in the art (see, for example, Green et al., “Production of Polyclonal Antisera,” in: Immunochemical Protocols, pages 1-5, Manson, ed., Humana Press. 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, such as syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal. Antibodies can also be derived from a subhuman primate antibody. General techniques for raising therapeutically useful antibodies in baboons can be found, for example, in PCT Publication No. WO 91/11465; and Losman et al., Int. J. Cancer 46:310, 1990.

Alternatively, an antibody that specifically binds an IaI polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from Stratagene Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody, defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Natl. Acad. Sci. U.S.A. 69:2659, 1972). Alternatively, the variable chains can be linked by an intermolecular disulfide bond or cross-linked by chemicals such as glutaraldehyde (see, for example, Sandhu, Crit. Rev. Biotech. 12:437, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin, thyroglobulin, bovine serum albumin, and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal antibody.

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, e.g., therapeutic, diagnostic, or detection moieties can be linked to an antibody that specifically binds IaI, using any number of means known to those of skill in the art. Exemplary effector molecules include, but not limited to, radiolabels, fluorescent markers, or toxins (e.g. Pseudomonas exotoxin, see “Monoclonal Antibody-Toxin Conjugates: Aiming the Magic Bullet,” Thorpe et al., “Monoclonal Antibodies in Clinical Medicine,” Academic Press, pp. 168-190, 1982; Waldmann, Science, 252: 1657, 1991; U.S. Pat. No. 4,545,985 and U.S. Pat. No. 4,894,443, for a discussion of toxins and conjugation). Both covalent and noncovalent attachment means may be used. The procedure for attaching an effector molecule to an antibody varies according to the chemical structure of the effector. Polypeptides typically contain a variety of functional groups (e.g., carboxylic acid (COOH), free amine (—NH₂) or sulfhydryl (—SH) groups), which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, label (e.g. enzymes or fluorescent molecules) drugs, toxins, and other agents to antibodies, one skilled in the art will be able to determine a suitable method for attaching a given agent to an antibody or other polypeptide.

### VII. Heparin Molecules as IaI Inhibitors

In one embodiment of the methods provided herein, the IaI inhibitor administered to the subject with an airway disease or disorder is a glycosaminoglycan molecule. Glycosaminoglycans contain multiple repeats of a basic disaccharide unit. Exemplary glycosaminoglycans include, but are not limited to chondroitin sulfate, heparan sulfates and heparin molecules. In some embodiments, the glycosaminoglycan is a heparin molecule. Heparin, a sulfated mucopolysaccharide, is synthesized in mast cells as a proteoglycan and is particularly abundant in the liver and lungs of various animals. Heparin is not a specific compound of fixed molecular weight but is actually a heterogenous mixture of variably sulfated polysaccharide chains composed of repeating units of D-glucosamine and either L-iduronic or D-glucuronic acids. The average molecular weight of heparin isolated from animal tissues ranges from about 6,000 to about 30,000 daltons (U.S. Pat. No. 5,690,910).

Pharmacologically, heparin is known primarily as an anticoagulant. This activity results from heparin's ability to bind to some of the residues of antithrombin III (AT-III), accelerating the neutralization by AT-III of activated clotting factors and preventing the conversion of prothrombin to thrombin. Larger amounts of heparin can inactivate thrombin and earlier clotting factors, preventing conversion of fibrinogen to fibrin.

The anticoagulant activity of heparin is related to the molecular weight of its polysaccharide fragments; low molecular weight components or fragments (for example, fragments having a molecular weight of less than 6,000 daltons) have moderate to low antithrombin and hemorrhagic effects. Similarly, low molecular weight heparins isolated from animal tissue have reduced anticoagulant properties because they consist primarily of the lower molecular weight fragments or fractions. Commercial heparin, which is generally derived from beef lung or pork intestinal mucosa, has an average molecular weight of about 15,000-17,500 daltons.

It has been reported that low molecular weight heparins (average molecular weight about 4,500 daltons), and ultra-low molecular weight heparin (ULMWH) fractions, were effective at reducing airway-hyperresponsiveness in animals following antigen challenge, while exhibiting reduced anticoagulant activity (U.S. Pat. Nos. 5,690,910; 5,980,865; 6,193,957; and 7,056,898). ULMWH is typically defined as heparin fractions having an average molecular weight of 3,000 daltons or less.

Accordingly, heparin molecules that inhibit IaI function or activity are contemplated for use as therapeutic agents for the treatment of airway diseases and disorders according to the methods described herein. Any type of heparin molecule or heparin derivative that inhibits IaI can be used, including, but not limited to, low molecular weight heparin and ULMWH. In some embodiments, the heparin molecule is less than about 30,000 daltons, less than about 20,000 daltons, less than about 15,000 daltons, less than about 6,000 daltons, less than about 4,500 daltons, less than about 3000 daltons, or less than about 2,500 daltons. In particular examples, the heparin molecule is about 1,000 to about 3,000 daltons; about 2,500 to about 4,500 daltons; about 2,500 to about 6,000 daltons; or about 6,000 to about 30,000 daltons.

In some examples, the heparin molecule inhibits IaI but exhibits little to no anticoagulant activity. A subject treated with heparin can be administered any suitable amount of a heparin composition. In some embodiments, the patient is administered about 0.05 to about 1.0 mg of heparin per kilogram of patient body weight in each dose of the composition. In particular examples, the patient is administered about 0.075 to about 0.75 mg/kg per dose. The dose heparin will vary depending on variety of factors, including the type of heparin molecule, the age, weight and general health of the subject being treated, and the disease or disorder being treated. One of skill in the art is capable of determining an appropriate dose of heparin.

### VIII. Antisense and Related Compounds Specific for IaI

Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites.

Another example of modulation of gene expression by target degradation is RNA interference (RNAi) using small interfering RNAs (siRNAs). RNAi is a form of antisense-mediated gene silencing involving the introduction of double stranded (ds)RNA-like oligonucleotides leading to the sequence-specific reduction of targeted endogenous mRNA levels. Another type of antisense compound that utilizes the RNAi pathway is microRNA. MicroRNAs are naturally occurring RNAs involved in the regulation of gene expression. However, these compounds can be synthesized to regulate gene expression via the RNAi pathway. Similarly, shRNAs are RNA molecules that form a tight hairpin turn and can be used to silence gene expression via the RNAi pathway. The shRNA hairpin structure is cleaved by the cellular machinery into siRNA.

Other compounds that are often classified as antisense compounds are ribozymes. Ribozymes are catalytic RNA molecules that can bind to specific sites on other RNA molecules and catalyze the hydrolysis of phosphodiester bonds in the RNA molecules. Ribozymes modulate gene expression by direct cleavage of a target nucleic acid, such as a messenger RNA.

Each of the above-described antisense compounds provides sequence-specific target gene regulation. This sequence-specificity makes antisense compounds effective tools for the selective modulation of a target nucleic acid of interest. In some embodiments provided herein, the target nucleic acid molecule is a nucleic acid molecule encoding an IaI polypeptide. In particular examples, the nucleic acid molecule encodes the IaI light chain or one of the five IaI heavy chains.

A. Antisense Compounds Targeting IaI Subunits

As taught herein, functional blockade of IaI prevents or inhibits the development of AHR in animal models of airway disease. Accordingly, provided herein is a method of treating an airway disease in a subject by administering an inhibitor of IaI. IaI is a molecule comprised of a single L chain, encoded by AMBP, and two heavy chains. There are five known heavy chain polypeptides, encoded by ITIH1, ITIH2, ITIH3, ITIH4 and ITIH5. Contemplated herein are antisense compounds that target any IaI subunit nucleic acid molecule, including AMBP, ITIH1, ITIH2, ITIH3, ITIH4 and ITIH5.

In some embodiments, expression of an IaI subunit is inhibited at least about 10%, at least about 25%, at least 50%, at least 75%, at least 90%, or at least 95% relative to a control (such as compared to an untreated subject, or expression prior to treatment). Thus, provided are methods of using antisense compounds that target an IaI subunit nucleic acid molecule to prevent, treat or ameliorate an airway disease or disorder in a subject. Any type of antisense compound that specifically targets and regulates expression of an IaI subunit is contemplated for use with the disclosed methods. Such antisense compounds include single-stranded compounds, such as antisense oligonucleotides, and double-stranded compounds, including compounds with at least partial double-stranded structure, including siRNAs, miRNAs, shRNAs and ribozymes. Methods of designing, preparing and using antisense compounds that specifically target a nucleic acid molecule encoding an IaI subunit are within the abilities of one of skill in the art.

Furthermore, sequences for IaI subunits are publicly available. Exemplary GenBank Accession Numbers and deposit dates for the IaI light chain gene (AMBP) and the IaI heavy chain genes (ITIH1, ITIH2, ITIH3, ITIH4 and ITIH5) are shown in Table 1. Each of the Genbank sequences listed below is herein incorporated by reference; the specific sequences are provided for reference only and are not intended to be limiting.

TABLE 1
GenBank Accession Numbers for IaI Subunit Genes

| Gene | Accession No. | Deposit Date |
|---|---|---|
| AMBP | NM_001633 | Mar. 24, 1999 |
| AMBP | AL137850 | Jan. 31, 2000 |
| AMBP | AK290837 | Oct. 12, 2007 |
| AMBP | X04494.1 | Apr. 21, 1993 |
| ITIH1 | NM_002215 | Mar. 24, 1999 |
| ITIH1 | AC006254 | Dec. 25, 1998 |
| ITIH2 | NM_002216 | Mar. 24, 1999 |
| ITIH2 | AL158044 | Mar. 6, 2000 |
| ITIH3 | NM_002217 | Sep. 12, 2000 |
| ITIH3 | AC006254 | Dec. 25, 1998 |
| ITIH4 | NM_002218 | Mar. 24, 1999 |
| ITIH4 | AC006254 | Dec. 25, 1998 |
| ITIH5 | NM_001001851 | Jun. 28, 2004 |
| ITIH5 | AL158044 | Mar. 6, 2000 |

Antisense compounds specifically targeting an IaI subunit nucleic acid molecule can be prepared by designing compounds that are complementary to an IaI subunit nucleotide sequence, particularly the an IaI subunit mRNA sequence. Antisense compounds targeting an IaI subunit need not be 100% complementary to the IaI subunit to specifically hybridize and regulate expression the target gene. For example, the antisense compound, or antisense strand of the compound if a double-stranded compound, can be at least 75%, at least 80%, at least 85%, at least 90%, at least 95%, at least 99% or 100% complementary to the selected IaI subunit nucleic acid sequence. Methods of screening antisense compounds for specificity are well known in the art (see, for example, U.S. Patent Application Publication No. 2003-0228689).

B. Generation of shRNA Constructs

Typically, shRNA is transcribed in cells from a DNA template as a single-stranded RNA molecule of approximately 50 to 100 bases in length. Complementary regions spaced by a small loop region cause the transcript to fold back on itself, forming a short hairpin in a manner analogous to natural microRNA. Recognition and processing by the RNAi machinery converts the shRNA into the corresponding siRNA.

There are several methods for generating shRNA constructs (see, for example, McIntyre and Fanning, BMC Biotechnol. 6:1, 2006, PCT Publication No. WO 2007/010840, and U.S. Patent Application Publication Nos. 2007-0231807 and 2007-014594). One strategy is a PCR-based approach in which a promoter sequence serves as the template. The hairpin sequence is contained in the reverse primer and PCR results in a cloning cassette comprising both promoter and hairpin. Correct amplicon production is dependent upon the sequence of the reverse primer.

Another method encompasses several techniques relating to primer extension. Each is based on the principle of a polymerase extending the 3′ end of overlapping oligonucleotides. In one example, the shRNA template is formed from two long partially complementary oligonucleotides of approximately equal length, overlapping at their 3′ ends (Unwalla et al., Nat. Biotechnol. 22(12):1573-1578, 2004; Zeng et al., Methods Enzymol. 392:371-380, 2005). Each oligonucleotide serves as both template (for extending the opposite oligonucleotide) and primer (to copy the opposite oligonucleotide). Extension and repeated cycling generates a double-stranded product, similar to that generated in the annealed oligonucleotide method. In a variation of this method, one long oligonucleotide is used as the template and a second short oligonucleotide (generic) is used as the primer for extension. The product can be further amplified by PCR with addition of another short primer binding the extended strand (Paddison et al., Nat Methods. 1:163-167, 2004).

The most common method for generating shRNA constructs involves the synthesis, annealing and ligation of two complementary oligonucleotides into an expression vector. A number of shRNA expression vectors systems are known in the art and are commercially available. For example, the BLOCK-iT™ adenoviral vector system (Invitrogen) allows for efficient delivery of shRNA sequences into a variety of cell types via transfection, transduction or infection with recombinant virus. The Knockout Inducible RNAi System (Clontech) allows for inducible of expression of a shRNA. Also available are the pDsiPHER™ vectors (MoleculA), which are designed to express shRNAs of approximately 59-61 nucleotides in length. Regardless of the expression vector used, the shRNA, once inside a cell, is processed into a siRNA of approximately 19 nucleotides in length, which modulates gene expression via the RNAi pathway.

The shRNA expression vectors can be virus-based vectors or plasmid vectors. In one embodiment, the vector is an adenovirus based vector. The vectors can express the shRNA constitutively or inducibly, depending on the promoter used to drive expression of the shRNA. In addition, shRNA vectors can be used for transient or stable transfection. The vectors can optionally include features such as reporter genes or selection markers (for example, antibiotic resistance). The expression vectors can be targeted to specific tissues via conjugation to a tissue-specific ligand. Alternatively, tissue-specific expression can be achieved using a tissue-specific promoter.

In some cases, the shRNA is encoded by a recombinant virus. The recombinant virus can be delivered to cells in vitro or to a subject in vivo. Targeted delivery of the recombinant virus to a particular tissue type can be achieved using any means known in the art. For example, the recombinant virus can be conjugated with a tissue-specific or cell-specific ligand. The ligand targets the recombinant virus to the cell or tissue expressing the receptor for the ligand. Alternatively, the recombinant virus can be selected based on the tissue types the virus normally infects (referred to as viral tropism). Also, the expression of the shRNA encoded by the recombinant virus can be driven by a tissue-specific promoter. In this case, additional tissue types may be infected with the virus, but the shRNA would only be expressed in the tissues in which the promoter is active.

C. Antisense Compound Modifications

In some examples, the antisense compounds described herein contain one or more modifications to enhance nuclease resistance and/or increase activity of the compound. Modified antisense compounds include those comprising modified backbones or non-natural internucleoside linkages. As defined herein, antisense compounds having modified backbones include those that retain a phosphorus atom in the backbone and those that do not have a phosphorus atom in the backbone.

Examples of modified oligonucleotide backbones include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of the nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In some embodiments, both the sugar and the internucleoside linkage of the nucleotide units of the antisense compound are replaced with novel groups. One such modified compound is an oligonucleotide mimetic referred to as a peptide nucleic acid (PNA). In PNA compounds, the sugar-backbone of an oligonucleotide is replaced with an amide containing backbone, in particular an aminoethylglycine backbone. The bases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al. (Science 254, 1497-1500, 1991).

Modified antisense compound can also contain one or more substituted sugar moieties. In some examples, the antisense compounds can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In other embodiments, the antisense compounds comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one example, the modification includes 2′-methoxyethoxy (also known as 2′-O-(2-methoxyethyl) or 2′-MOE) (Martin et al., Hely. Chim. Acta. 78, 486-504, 1995). In other examples, the modification includes 2′-dimethylaminooxyethoxy (also known as 2′-DMAOE) or 2′-dimethylaminoethoxyethoxy (also known in the art as 2′-O-dimethylaminoethoxyethyl or 2′-DMAEOE).

Similar modifications can also be made at other positions of the compound. Antisense compounds can also have sugar mimetics such as cyclobutyl moieties in place of the pentofuranosyl sugar. Representative United States patents that teach the preparation of modified sugar structures include, but are not limited to, U.S. Pat. Nos. 4,981,957; 5,118,800; 5,319,080; 5,359,044; 5,393,878; 5,446,137;

5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Antisense compounds can also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further modified bases have been described (see, for example, U.S. Pat. No. 3,687,808; and Sanghvi, Y. S., Chapter 15, Antisense Research and Applications, pages 289-302, Crooke, S. T. and Lebleu, B., ed., CRC Press, 1993). Certain of these modified bases are useful for increasing the binding affinity of antisense compounds. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. Representative U.S. patents that teach the preparation of modified bases include, but are not limited to, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692.

D. Aerosol Delivery of Antisense Compounds

Antisense compounds can be delivered using any suitable route of administration. As described herein, local delivery of IaI inhibitors to the airway (such as the lung) is often desirable to avoid side effects that may occur following systemic administration. Pulmonary administration of antisense compounds results in minimal systemic exposure, potentially increasing the safety of such compounds as compared to other classes of therapeutic agents. In addition, the lung provides an ideal tissue for aerosolized antisense compounds for several reasons, including: the lung can be targeted non-invasively and specifically, it has a large absorption surface; and it is lined with surfactant that may facilitate distribution and uptake of antisense compounds (Nyce and Metzger, Nature, 1997: 385:721-725), Delivery of antisense compounds to the lung by aerosol results in excellent distribution throughout the lung in both mice and primates Immunohistochemical staining of inhaled antisense compounds (such as antisense oligonucleotides) in normalized and inflamed mouse lung tissue shows heavy staining in alveolar macrophages, eosinophils, and epithelium, moderate staining in blood vessels endothelium, and weak staining in bronchiolar epithelium.

Compositions and methods for formulation of antisense compounds and devices for delivery to the lung and nose are well known (see, for example, U.S. Patent Application Publication No. 2008/0103106). Antisense compounds are soluble in aqueous solution and can be delivered using standard nebulizer devices (Nyce, Exp. Opin. Invest. Drugs 6:1149-1156, 1997). Formulations and methods for modulating the size of droplets using nebulizer devices to target specific portions of the respiratory tract and lungs are well known to those skilled in the art. Antisense compounds can be delivered using other devices such as dry powder inhalers or metered dose inhalers which can provide improved patient convenience as compared to nebulizer devices, resulting in greater patient compliance.

The antisense compounds and therapeutic compositions thereof can be administered in a number of ways depending upon whether local or systemic treatment is desired and upon the area to be treated. In some embodiments, administration is topical to the surface of the respiratory tract, particularly pulmonary (e.g., by nebulization, inhalation, or insufflation of powders or aerosols, by mouth and/or nose).

The pharmaceutical formulations of the present disclosure, which may conveniently be presented in unit dosage form, can be prepared according to conventional techniques well known in the pharmaceutical industry. Such techniques include the step of bringing into association the active ingredients (including the antisense compound targeting an IaI subunit nucleic acid molecule) with the pharmaceutical carriers or excipients. In general, the formulations are prepared by uniformly and intimately bringing into association the active ingredients with liquid carriers, finely divided solid carriers, or both, and then, if necessary, shaping the product (e.g., into a specific particle size for delivery). In some embodiments, the compositions comprising the antisense compounds of the instant disclosure are prepared for pulmonary administration in an appropriate solvent (e.g., water or normal saline), possibly in a sterile formulation, with carriers or other agents to allow for the formation of droplets of the desired diameter for delivery using inhalers, nasal delivery devices, nebulizers, and other devices for pulmonary delivery. Alternatively, the pharmaceutical formulations of the instant invention may be formulated as dry powders for use in dry powder inhalers.

### IX. Identification of Inhibitors of IaI

As described herein, the inhibitor of IaI can be any type of molecule that serves as a pharmacological inhibitor of the IaI protein or a nucleic acid encoding an IaI subunit. Methods of screening candidate therapeutic agents to identify an inhibitor of a target molecule are well known in the art. Thus, it is within the skill of the ordinary artisan to design and carry out an assay to screen for therapeutic agents that inhibit expression or activity of IaI or an IaI subunit. An “agent” is any substance or any combination of substances that is useful for achieving an end or result; for example, a substance or combination of substances useful for modulating gene expression or protein activity. Any agent that has potential to modulate IaI expression or activity is contemplated for use in the methods of this disclosure.

A. Compound Libraries

The use of antibodies, heparin or antisense compounds as inhibitors of IaI is described in detail above. Additional exemplary candidate inhibitors include, but are not limited to, peptides such as, for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries (see, e.g., Songyang et al., Cell, 72:767-778, 1993), small organic or inorganic molecules (such as, so-called natural products or members of chemical combinatorial libraries), molecular complexes (such as protein complexes), or nucleic acids.

Libraries (such as combinatorial chemical libraries) useful for identifying candidate IaI inhibitors include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; International Publication No. WO 91/19735), encoded peptides (e.g., International Publication WO 93/20242), random bio-oligomers (e.g., International Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996), carbohydrate libraries (see, e.g., Liang et al., Science, 274:1520-1522, 1996; U.S. Pat. No. 5,593,853), small organic molecule libraries (see, e.g., benzodiazepines, Baum, C&EN, January 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries useful for identifying candidate IaI inhibitors can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds. Such combinatorial libraries are then screened in one or more appropriate assays to identify those library members (particularly chemical species or subclasses) that display a desired characteristic activity (such as, inhibition of expression or activity of IaI or an IaI subunit). The compounds thus identified can serve as conventional “lead compounds” or can themselves be used as potential or actual therapeutics. In some instances, pools of candidate inhibitors may be identify and further screened to determine which individual or subpools of candidate inhibitors in the collective have the desired activity.

B. Screening Assays

Identification of IaI inhibitors can be achieved using any suitable assay for detecting inhibition of IaI expression or activity. The IaI heavy chain polypeptides are known to bind hyaluronan. It is disclosed herein that hyaluronan mediates AHR in an animal model of airway disease. Thus, an exemplary screening assay to identify an IaI inhibitor includes an assay for identifying agents that inhibit binding of IaI to hyaluronan. This type of screening assay can evaluate binding activity of IaI to hyaluronan in the presence and absence of a candidate agent using any suitable binding assay. Binding assays are well known in the art and include, for example, enzyme-linked immunosorbent assays (ELISA), yeast two-hybrid assays and immunoprecipitation assays. In particular examples, IaI and hyaluronan are incubated (such as in a cell-free system) in the presence or absence of a candidate agent. IaI binding to hyaluronan is evaluated using an ELISA according to standard procedures. A decrease in binding of IaI to hyaluronan in the presence of the candidate agent, relative to binding in the absence of the candidate agent, indicates the candidate agent is an IaI inhibitor. IaI inhibitors identified using this type of assay can be further evaluated in an appropriate animal model of airway disease (such as a one of the models described herein) to determine whether the IaI inhibitor is useful for the treatment of an airway disease or disorder.

In addition, any of the methods described in the Examples below can be used to identify agents that inhibit IaI, for example the ozone, LPS and OVA animal models described in Example 8.

The following examples are provided to illustrate certain particular features and/or embodiments. These examples should not be construed to limit the disclosure to the particular features or embodiments described.

### EXAMPLES

### Example 1

### Experimental Procedures

This example describes the methods used for the studies described in Examples 2-7.

Mice: CD44-deficient (CD44−/−) mice were backcrossed onto C57BL/6J for greater than 10 generations (Schmits et al., Blood 90(6):2217-2233, 1997). Bikunin/IaI-deficient (IaI−/−) mice also were backcrossed onto C57BL/6J for greater than 10 generations (Zhuo et al., J Biol Chem 276(11):7693-7696, 2001). CC10-HAS2 transgenic animals were backcrossed onto C57BL/6J background for greater than 5 generations (Jiang et al., Nat Med 11(11):1173-1179, 2005). Experimental groups consisted of 10 male, 6 to 8 week-old mice unless otherwise stated.

Exposure Protocol: C57BL/6J, CD44−/−, IaI−/− or HAS2 transgenic mice (mice that overexpress HAS2) were exposed to either Hepa-filtered air (FA) or ozone. Animals were housed in cages with low-endotoxin bedding, and given water and chow ad libitum. Ozone exposures were two parts per million (ppm) for three hours. The selection of ozone concentration levels in the mouse was based on similar biological responses observed in human exposure studies and published deposition fraction data for 0₃ in rodent models (Wiester et al., Toxicol Appl Pharmacol 96(1):140-146, 1988). Exposures were performed in 55-liter Hinner chambers with individual animal slots. Air at 20-22° C. and 50-60% relative humidity was supplied at 20 exchanges per hour. Ozone was generated by directing 100% O₂ through an UV light generator and mixed with air supply to the chamber. Chamber ozone concentration was monitored continuously with a UV light photometer (1003AH, Dasibi, Glendale, Calif.). In some experiments, C57BL/6 mice were given 10 mg/kg subcutaneous hyaluronan binding protein (HABP) or scrambled binding protein control (SBP) (Savani et al., Am J Respir Cell Mol Biol 23(4):475-484, 2000) one hour prior to exposure. In other experiments, IaI−/− mice were injected intraperitoneally with 1 ml of 0.5 mg/ml IaI (ProThera, East Providence, R.I.) or 1 ml of 1 mg/ml urinary trypsin inhibitor/bikunin (GenScript, Piscataway, N.J.) 1 hour prior to ozone exposure.

HA challenge: Sterile, endotoxin-free (0.00008 EU/ml) high molecular weight hyaluronan (HMW-HA) (Healon, AMO, Santa Ana, Calif.) was reconstituted at 0.5 mg/ml in 0.02 M acetate/0.15 M sodium chloride, pH 6.0. For the production of low molecular weight (also known as short-fragment) hyaluronan (sHA), healon was sonicated on ice. Sizes were confirmed by electrophoresis according to known methods (Lee and Cowman Anal Biochem 219(2):278-287, 1994). In some experiments, 50 μl of HMW-HA, sHA, or vehicle were instilled oropharyngeally into isoflurane-anesthetized mice, and AHR was measured invasively 2-4 hours later. In other experiments, 50 μl of HMW-HA or vehicle was instilled 1 hour before and 23 hours after acute ozone exposure, and AHR was measured invasively 24 hours after ozone exposure.

Airway Physiology: Twenty four hours after the beginning of exposure, tracheas of anesthetized mice (pentobarbital sodium, 60 mg/kg i.p.) were surgically dissected and intubated, mice were paralyzed (pancuronium bromide, 0.08 μg/kg i.v.) and ventilated with a computer-controlled small animal ventilator (FlexiVent™, SCIREQ, Montreal, Canada) at a tidal volume of 7.5 ml/kg and a positive end-expiratory pressure of 3 cm H₂O. Forced oscillation was used for measurements of respiratory mechanics. Briefly, airway pressure and tidal volume data were generated by the application of a 2-s sine wave volume perturbation with 0.2 ml amplitude and 2.5 Hz frequency. Following base-line resistance measurements, mice were challenged with methacholine aerosol (DeVilbiss ultrasonic) at 0, 10, 25, and 100 mg/ml. Between aerosol doses, the lung was hyperinflated with total lung capacity breath to return resistance to baseline levels. Total lung resistance measurements were averaged at each dose and graphed (R_{T} cmH₂O/ml/s) along with the initial baseline measurement.

Whole lung lavage: Whole lung lavage was performed according to previously described methods (Hollingsworth et al., Am J Respir Crit Care Med 170(2):126-132, 2004). Supernatants were stored at −70° C.

Hyaluronan measurements: Lavage hyaluronan levels were performed with a commercially available ELISA (Echelon, San Jose, Calif.) as per manufacturer's instructions. For electrophoresis, lavage fluid was incubated 1:1 (v/v) with protease (Sigma, St Louis. Mo.), at 37° C. for 24 hours, then boiled for 10 minutes, quenched on ice, centrifuged, and the supernatant was concentrated 10× in a vacuum centrifuge. Concentrates were run on a 0.5% agarose gel, together with Healon, sonicated Healon, and hyaluronan ladders (Hyalose, Oklahoma City, Okla.), stained overnight with 0.005% Stains-All (Sigma, St Louis, Mo.) in 50% ethanol, and then de-stained in distilled water and photographed.

Immunohistochemistry: Formalin-fixed lungs were sectioned in 5 μm thick sections, and stained with PE-Cy5-conjugated anti-mouse CD44 (BD Pharmingen, San Jose, Calif.) and biotinylated Hyaluronan-binding protein (HABP) (Seikagaku Corp. Associates of Cape Cod, Fallmouth, Mass.). A secondary streptavidine-Alexa 488 fluorochrome (Invitrogen, Carlsbad, Calif.) was used to detect hyaluronan.

Statistics: Data are expressed as mean±SEM. Significant differences between groups were identified by ANOVA and the Student t-test unless otherwise stated. A two-tailed P value of <0.05 was considered statistically significant.

### Example 2

### Ozone Exposure Increases Hyaluronan Concentration in Mouse Lung Lavage Fluid

This example describes the finding that hyaluronan is detected in lavage fluid and the subepithelial space of animals following exposure to ozone.

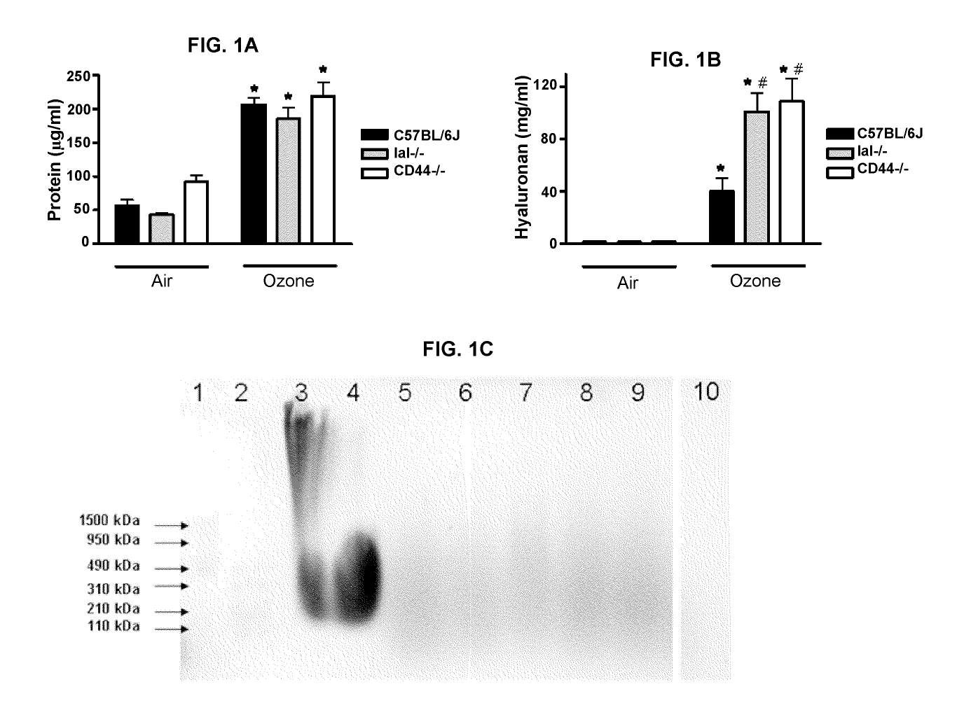

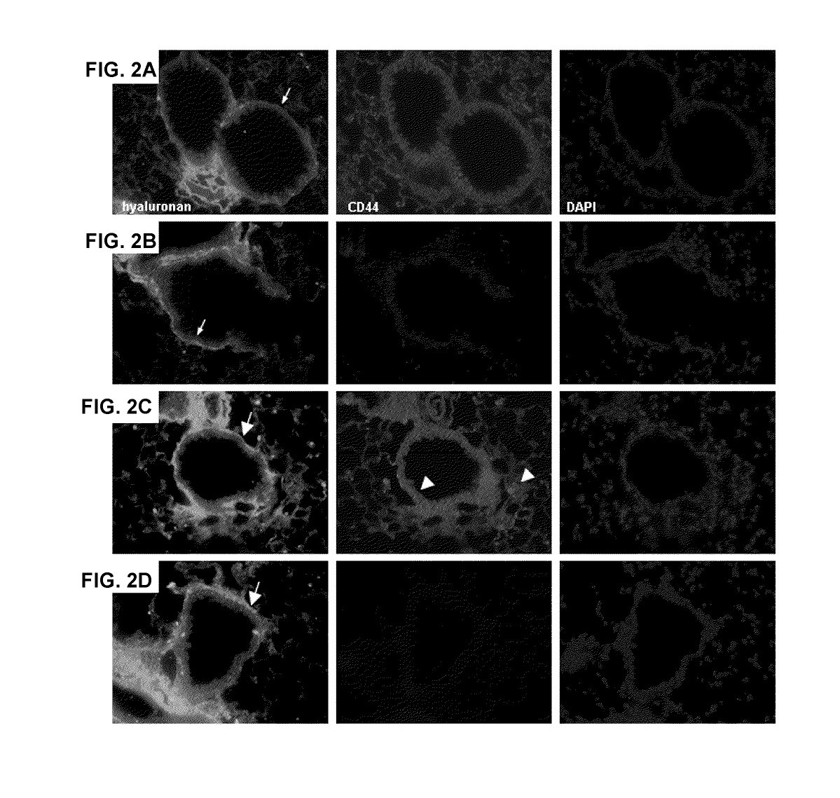

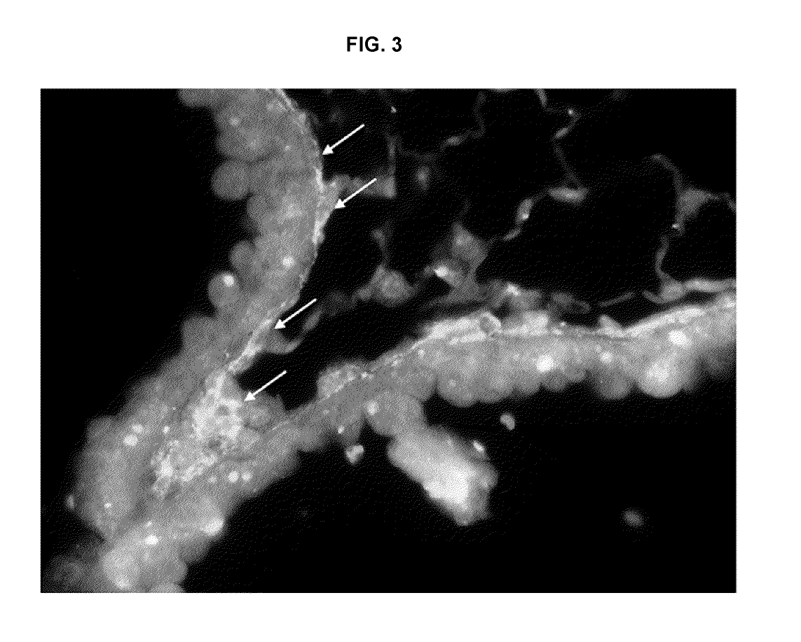

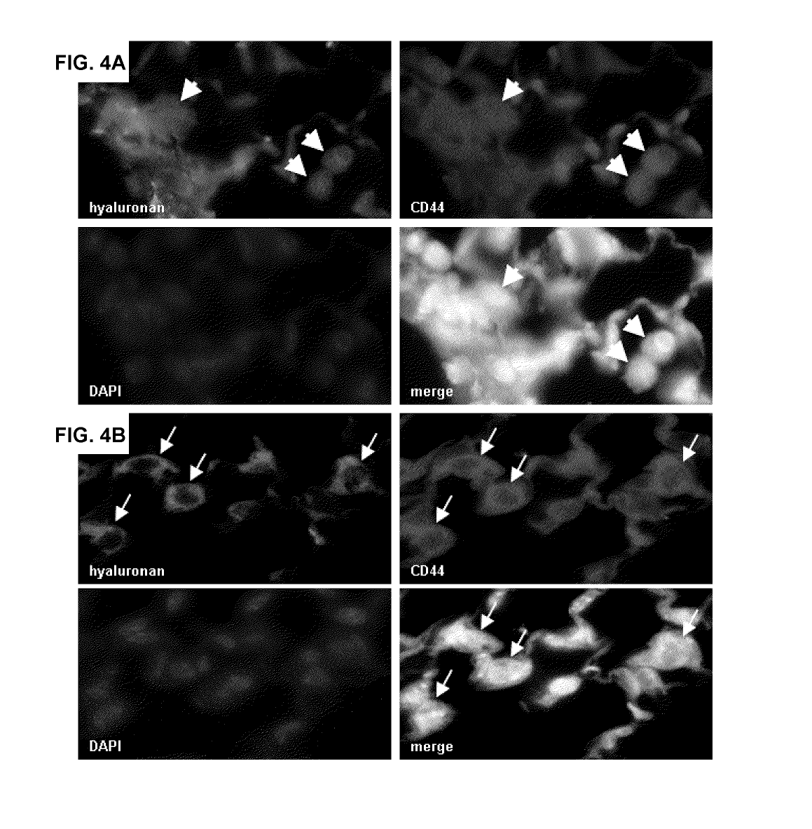

C57BL/6 mice, CD44-deficient mice, and IaI-deficient mice were exposed to 2 ppm ozone for 3 hours. The level of airway injury as measured by lavage protein was similar in all ozone-exposed groups and increased when compared to filtered air-exposed (control) mice (FIG. 1A). Exposure to ozone increased the levels of soluble hyaluronan in bronchial alveolar lavage fluid of all strains of mice (FIG. 1B). Consistent with the role of CD44 and IaI in clearance of free hyaluronan, enhanced levels of HA in CD44−/− and IaI−/− when compared to wild-type mice were observed. Soluble hyaluronan in the lavage fluid was of lower molecular weight, averaging about 1 kDa (sHA) (FIG. 1C). After ozone exposure, the hyaluronan receptor CD44 was detected on both the airway epithelia and alveolar macrophages by fluorescent microscopy. Hyaluronan was primarily visible in the subepithelial space, where there was increased hyaluronan deposition after ozone exposure (FIG. 2). Hyaluronan was particularly visible around subepithelial myocytes (FIG. 3), while CD44 and hyaluronan co-localized on alveolar macrophages (FIG. 4). Cumulatively, these observations suggest that ozone exposure can release hyaluronan in both the lavage fluid and subepithelial space, where different cell-types could bind hyaluronan and mediate hyaluronan-induced effects.

### Example 3

### HA Recognition is Required for the Development of AHR After Ozone Exposure

This example describes the finding that CD44 and IaI are required for the physiological response to ozone.

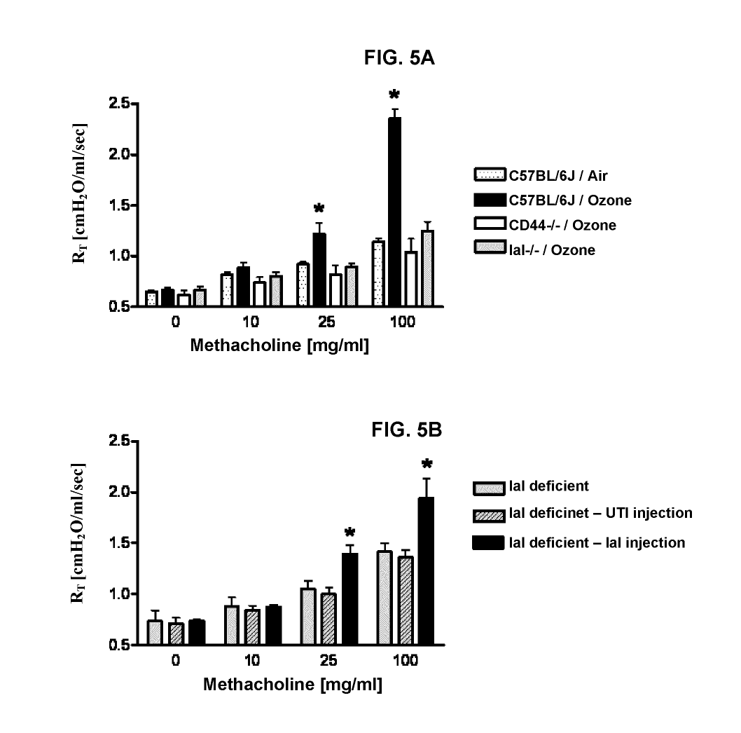

To determine the role of known receptors of hyaluronan in the biologic response to ozone, CD44-deficient mice were characterized. It was determined that these mice were protected from ozone-induced airway hyperresponsiveness, when compared to C57BL/6J mice (FIG. 5A). Furthermore, the role of IaI, which facilitates hyaluronan-dependent signaling (Zhuo et al., J Biol Chem 281(29):20303-20314, 2006), was examined Consistent with the findings in CD44−/−, IaI−/− animals were also protected from the physiologic response to ambient ozone (FIG. 5A). IaI consists of two heavy chains, which can bind hyaluronan, and a light chain called urinary trypsin inhibitor (UTI)/bikunin, which is an anti-inflammatory protease inhibitor, but does not bind hyaluronan (Zhuo et al., J Biol Chem 279(37):38079-38082, 2004).

Therefore, it was tested whether the hyaluronan-binding component of IaI was necessary to mediate ozone-induced AHR. Intraperitoneal injection of IaI, but not UTI/bikunin, into IaI-deficient mice reconstituted the physiologic response to ozone (FIG. 5B). This observation supports an essential role of IaI in the biologic response to ozone and provides further evidence for the role of hyaluronan in mediating ozone-induced AHR. These observations demonstrate that the biological response to ozone is, in part, dependent on both CD44 and IaI. Each of these genes is known to be involved in the cellular recognition of hyaluronan, further supporting the role of this molecule in the functional response to ozone.

### Example 4

### HA-Binding Attenuates Ozone Induced Airway Hyperresponsiveness

This example describes the finding that hyaluronan binding protein (HABP) significantly attenuates ozone-induced AHR.

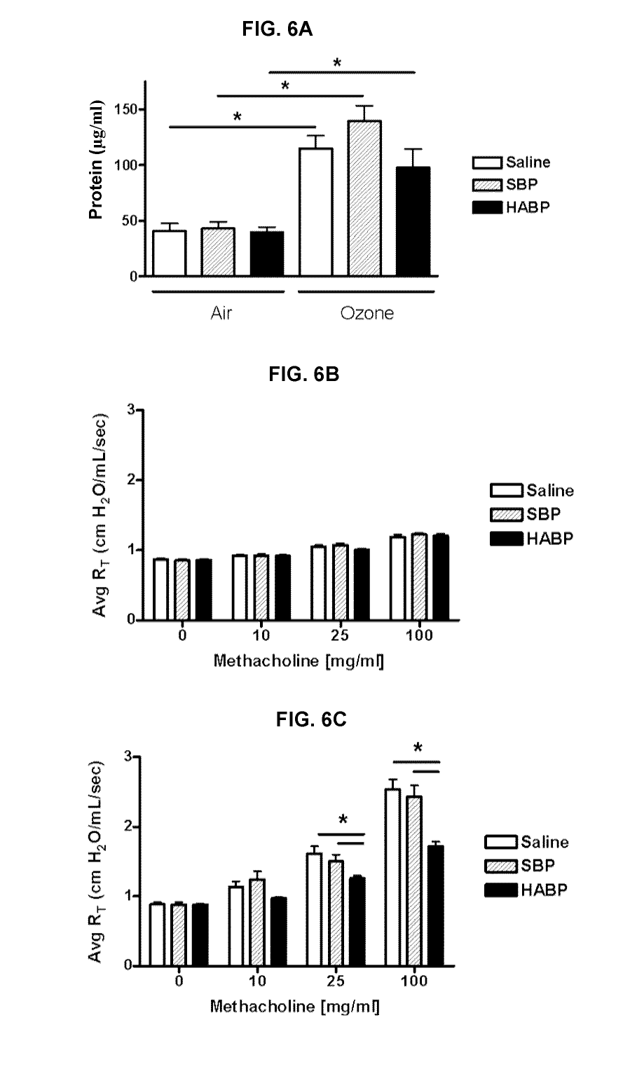

To directly determine the role of hyaluronan in airway hyperresponsiveness to ozone, C57BL/6J animals were treated with HABP before ozone exposure. This reagent has previously been used to bind HA and attenuate the biological effects of this molecule (Savani et al., Am J Respir Cell Mol Biol 23(4):475-484, 2000). There was no difference in the degree of lung injury as quantified by lung lavage protein levels (FIG. 6A). Treatment with either HABP or SBP had no effect on baseline AHR (FIG. 6B). It was observed that neutralization of hyaluronan with HABP significantly attenuated ozone-induced AHR similar to baseline, when compared to either scrambled binding peptide (SBP) or untreated mice (FIG. 6C). Similar results were obtained after exposure to sub-chronic low dose ozone (0.3 ppm for 72 hours). In those experiments, HABP, but not SPB, attenuated ozone-induced AHR to the level seen in CD44−/− mice and unexposed animals. These observations further support the role of HA in the biological response to ozone.

### Example 5

### Overexpression of Hyaluronan Enhances Ozone-Induced AHR

This example describes the finding that ozone-induced modification of HA is required to induce AHR.

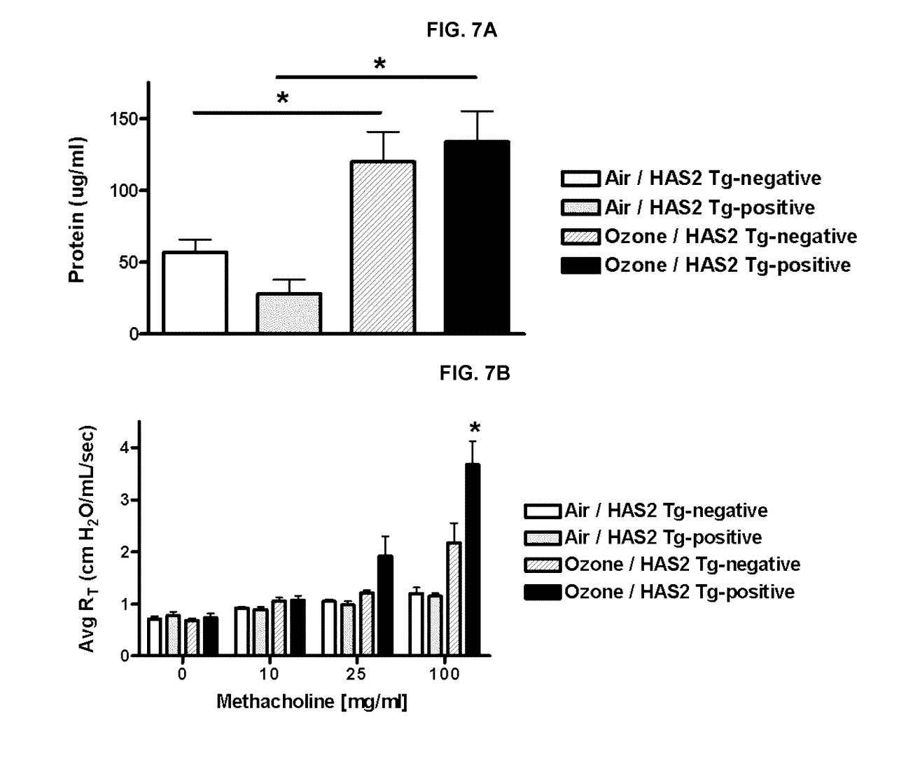

To determine the role of increased levels of hyaluronan in the response to ozone, transgenic mice that over-express hyaluronan synthase 2 (HAS2) by airway epithelia resulting in enhanced production of HMW-HA (Jiang et al., Nat Med 11(11):1173-1179, 2005), were exposed to ozone. Despite increased soluble levels of HMW-HA, a similar degree of lung injury as quantified by lung lavage protein levels was observed (FIG. 7A). There was no difference in AHR observed between transgene-negative and transgene-positive animals with filtered air exposure. After ozone exposure, transgene-negative littermate mice demonstrated the expected enhanced response to methacholine after exposure to ozone. However, transgene-positive animals demonstrated an exaggerated AHR response after ozone challenge (FIG. 7B). Cumulatively, these observations support that the elevated level of HMW-HA alone is not sufficient to cause AHR and suggest that ozone-induced modification of HA are required to induce AHR.

### Example 6

### Short-Fragment HA (sHA) is Sufficient to Induce Airway Hyperresponsiveness

This example describes the finding that sHA can induce airway hyperresponsiveness, while HMW-HA has a protective role in ozone-induced AHR.

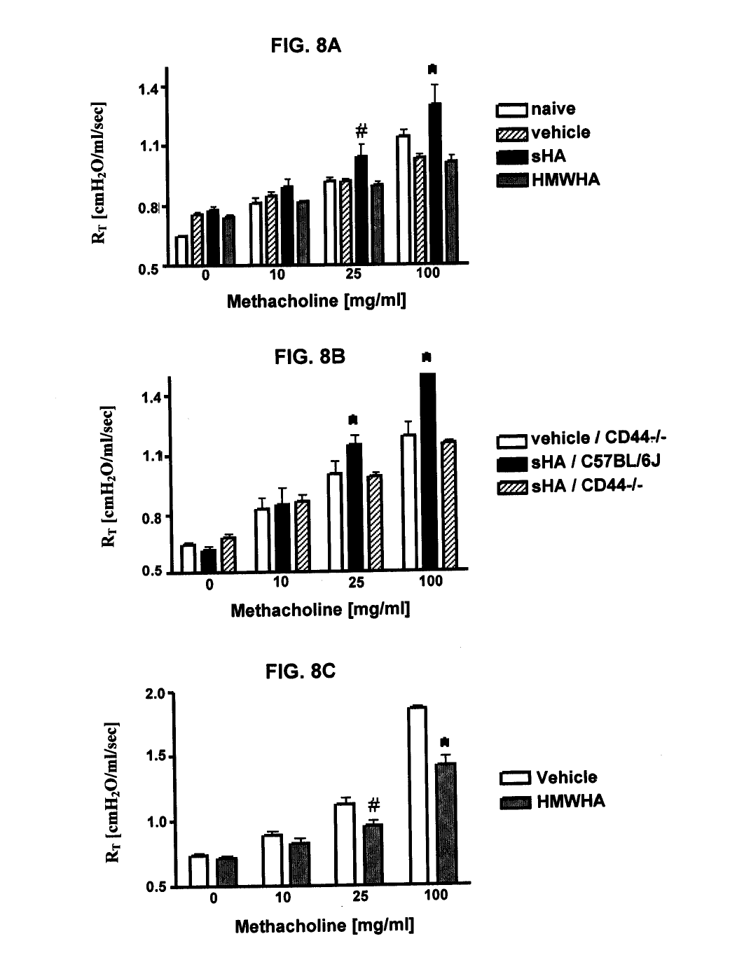

CD44 and IaI can each bind to hyaluronan, and recent evidence supports that sHA mediates non-infectious lung injury (Jiang et al., Nat Med 11(11):1173-1179, 2005). To specifically address the role of sHA in airway hyperresponsiveness, naïve mice were directly challenged by oropharyngeal aspiration of hyaluronan. Endotoxin-free HMW-HA was sonicated to create sHA of similar size as hyaluronan in lung lavage fluid after ozone exposure (FIG. 1C). Instillation of sHA, but not HMW-HA, into naive C57BL/6J mice induced AHR when compared to vehicle (FIG. 8A). The surface receptor CD44 was necessary for this hyaluronan-dependent AHR (FIG. 8B). These data indicate that sHA can, in part, mimic ozone-induced AHR. Interestingly, significant changes in total cells or cell composition in the lung lavage fluid were not observed after sHA instillation, suggesting that the sHA effect on AHR may not require the presence of recruited inflammatory cells. To further corroborate this finding, mice were treated with HMW-HA before and after ozone challenge, since HMW-HA can competitively inhibit sHA effects (Deed et al., Int J Cancer 71(2):251-256, 1997). A significant attenuation of AHR in mice treated with HMW-HA concomitantly with ozone exposure was observed (FIG. 8C). Cumulatively, these findings demonstrate that sHA can induce airway hyperresponsiveness and that HMW-HA has a protective role in ozone-induced AHR, supporting that hyaluronan size is an important factor in ozone-induced AHR.

### Example 7

### Inflammatory Cell Migration Into the Lungs is Dependent on Hyaluronan Binding Through CD44 and IaI

This example describes the finding that CD44 and IaI play an important role in recruitment of inflammatory cells to the lung during ozone-induced AHR.

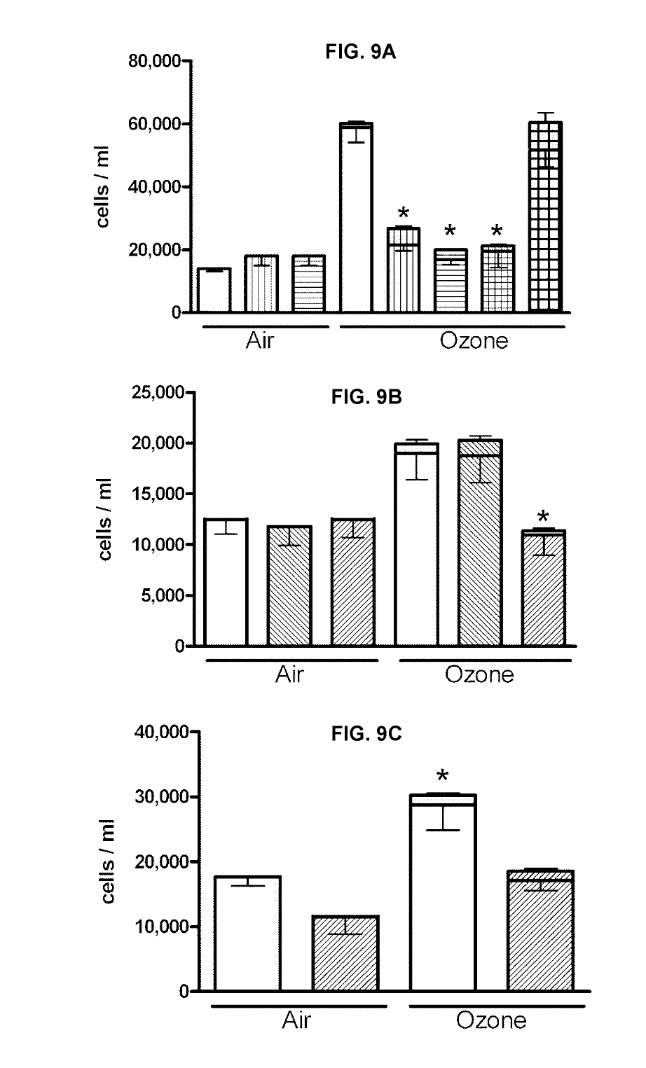

CD44 and IaI can either negatively or positively modify cellular inflammation in the lung depending on the severity of lung injury, as well as the environmental stimuli. Inflammation in the lung has also been associated with the severity of AHR. It was therefore essential to characterize the severity of alveolar inflammation after exposure to ozone in CD44−/−, and IaI−/− mice. For both CD44-deficient and IaI-deficient mice, decreased total cell counts in whole lung lavage fluid were observed at 24 hours after exposure (the time of maximum AHR) when compared to C57BL/6 mice. Macrophages accounted for most of the observed differences. Recruitment of inflammatory cells was rescued in IaI−/− mice through pre-injection of IaI, but not UTI/bikunin (FIG. 9A). This finding is consistent with the idea that CD44-IaI-mediated inflammatory cell migration is dependent on hyaluronan binding. Both CD44 and Id have been described to mediate cell binding to hyaluronan (Zhuo et al., J Biol Chem 281(29):20303-20314, 2006) and CD44 plays a role in endothelial adhesion of monocytes (Hollingsworth et al., Am J Respir Cell Mol Biol, 2007). In the hyaluronan-binding experiments, a significant decrease of inflammatory cells (mainly macrophages) was observed in the lavage fluid of HABP-treated mice, but not SBP-treated mice (FIG. 9B). By contrast, a decrease in inflammatory cells in ozone-treated HAS2 transgene-positive mice, compared to ozone-treated controls was found (FIG. 9C). Finally, a significant change was not observed in any cell type in the alveolar compartment after exposure to sHA when compared to vehicle. These observations support an important role of CD44 and IaI in recruitment of inflammatory cells to the lung after exposure to ozone, but also suggest that the role of hyaluronan in airway hyperreactivity and cell recruitment is complex.

### Example 8

### Functional Blockade of IaI in Animal Models of Airway Disease

This example describes the finding that antibody-mediated blockade of IaI significantly reduces airway hyperresponsiveness in three different animal models following exposure to either ozone (O₃), lipopolysaccharide (LPS) or ovalbumin (OVA). The studies described below provide three distinct models for inflammation and AHR. The ozone exposure model is representative of environmental airway injury. In models of inhaled endotoxin (LPS), Th1 immune responses lead to AHR. The inhaled OVA model is a well-established model of allergic asthma, where Th2 immune responses lead to AHR. This example further demonstrates that animals deficient for IaI exhibit significantly reduced AHR in response to LPS.

### Acute Ozone (O₃) Exposure

Mice were placed individually in stainless steel cages during the exposure. Cages were set inside one of two separate 55 liter Hinners-style exposure chambers situated inside a fume hood. The exposure chambers were equipped with a charcoal-filtered and high-efficiency particulate filtered air supply. Chamber air was renewed at the rate of 20 changes per hour, with 50-65% relative humidity and a temperature of 20-25° C. Ozone was generated by directing a 100% oxygen gas source through an ultraviolet light ozone generator that was upstream from one of the exposure chambers. The Ozone-oxygen mixture was metered into the inlet air-stream and ozone concentrations were monitored regularly at different levels within the chamber with an ozone ultraviolet light photometer (Dasibi model 1003AH). Mice from each strain were exposed to 1.0 ppm ozone for 3 hours and then put in room air for 24 hours for recovery. Mice assigned to corresponding control groups were exposed to filtered air in the inhalation chambers for the same duration. After the 24 hour recovery period, mice underwent flexiVent™ (airway resistance measurement) followed by euthanasia and whole lung lavage and tissue collection. Antibodies were administered prior to flexiVent™ procedure.

### Inhaled Endotoxin (LPS):

In LPS for aerosolization was purchased as lyophilized, purified Escherichia coli 0111:B4 (Sigma, St. Louis, Mo.). LPS aerosol was generated and directed into a glass 20-liter exposure chamber using a Collison nebulizer (BGI Inc., Waltham, Mass.). High-efficiency particle apparatus-filtered air was supplied to the nebulizer at a constant pressure of 20 psi. The chamber atmosphere was exchanged at a rate of 0.25-1.0 changes per minute. LPS concentration was determined by sampling the total chamber outflow, 4-7 mg/m³. Mice were exposed to LPS at a dose of 5 μg/m³ for 2.5 hours. Mice had a recovery period of 1.5 hours post exposure before flexiVent™ (airway resistance measurement) and subsequent necropsy with whole lung lavage and tissue collection. Antibodies were administered prior to flexiVent™ procedure. A similar procedure was carried out on animals deficient for IaI to assess the effect of the absence of IaI on AHR.

### Ovalbumin (OVA) Asthma Model

Mice were sensitized by intraperitoneal injections of 10 μg OVA (100 μl) complexed with alum on days 1 and 7. On day 14, mice were exposed in a Hinner-style chamber to aerosolized OVA (1% W/V in PBS) for 20 minutes using a TSI 6-jet nebulizer set to deliver particles of approximately 0.2 μm. Forty-eight hours later, mice were subjected to flexiVent™ (airway resistance measurement) and subsequent necropsy with whole lung lavage and tissue collection. Antibodies were administered prior to flexiVent™ procedure.

### Antibody-Mediated Blockade of Inter-Alpha-Trypsin Inhibitor (IaI)

In each animal model, monoclonal (MAb 69.26) and polyclonal blocking antibodies (see U.S. Pat. No. 6,660,482; U.S. Patent Application Publication No. 2007/0297982; Lim et al., J. Infect. Dis. 188:919-926, 2003) specific for IaI were tested for their effect on airway hyperresponsiveness following exposure to ozone, LPS or OVA. MAb 69.26 binds the IaI light chain, bikunin, while the polyclonal antibody binds all components of IaI (heavy and light chains). Antibody was administered at a concentration of 1 mg/ml in 50 μl of PBS into the lung (oropharyngeal aspiration), 1 hour prior to flexiVent™ (i.e. 1.5 hours after exposure to LPS, 47 hours after exposure to OVA, and 23 hours after exposure to ozone).

Airway Responsiveness Measurements (flexiVent™)

Mice were anesthetized with intraperitoneal injection of pentobarbital sodium (60 mg/kg). The volume for a typical 25 gram mouse is approximately 80 μl. A surgically inserted tracheostomy cannula was used to ventilate the animals (6-8 ml/kg and 125 breaths/minute) and a differential pressure transducer was connected to the side ports of the tracheal cannula for the measurement of airway pressure. Tracheostomy was preformed by a small vertical midline incision followed by a horizontal incision into the trachea. Sodium pentobarbital and doxacurium chloride (0.25 mg/kg or 25 μl for a 25 gram mouse) were administered through a venous catheter to anesthetize and paralyze each mouse respectively. Mouse depth of anesthesia was checked by heart rate response to a toe pinch immediately before administering doxacurium chloride and monitored throughout surgery by changes in tracheal pressure and heart rate not in response to drug challenges. Increasing concentrations of aerosolized methacholine were used to challenge mice. Methacholine was administered with at least 5 minute recovery periods at three doses (5 mg/ml, 20 mg/ml, 100 mg/ml).

### Results

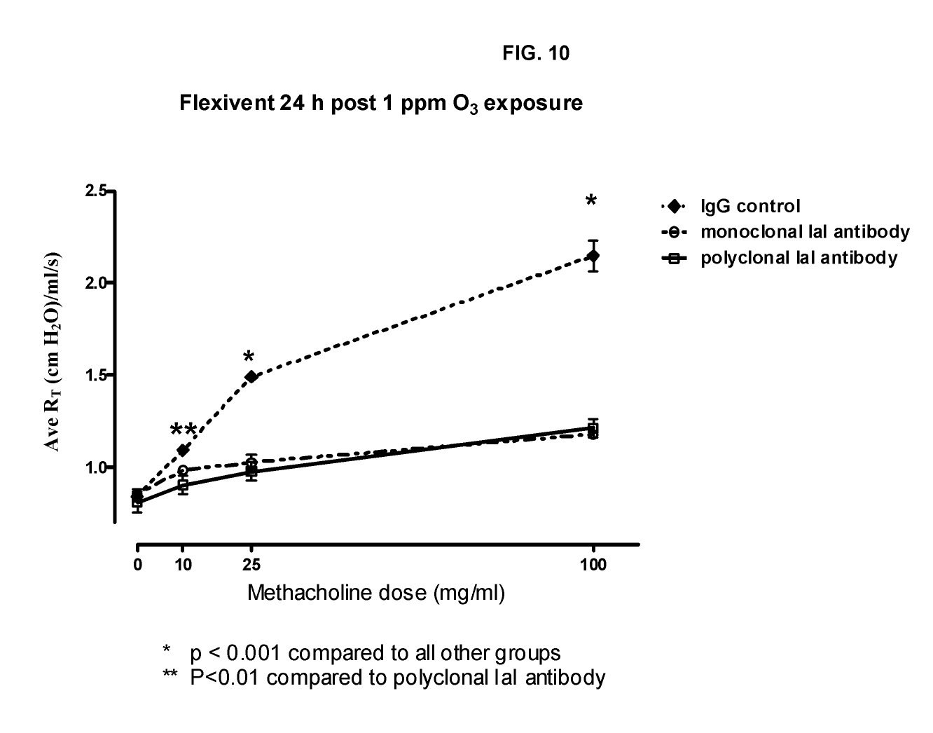

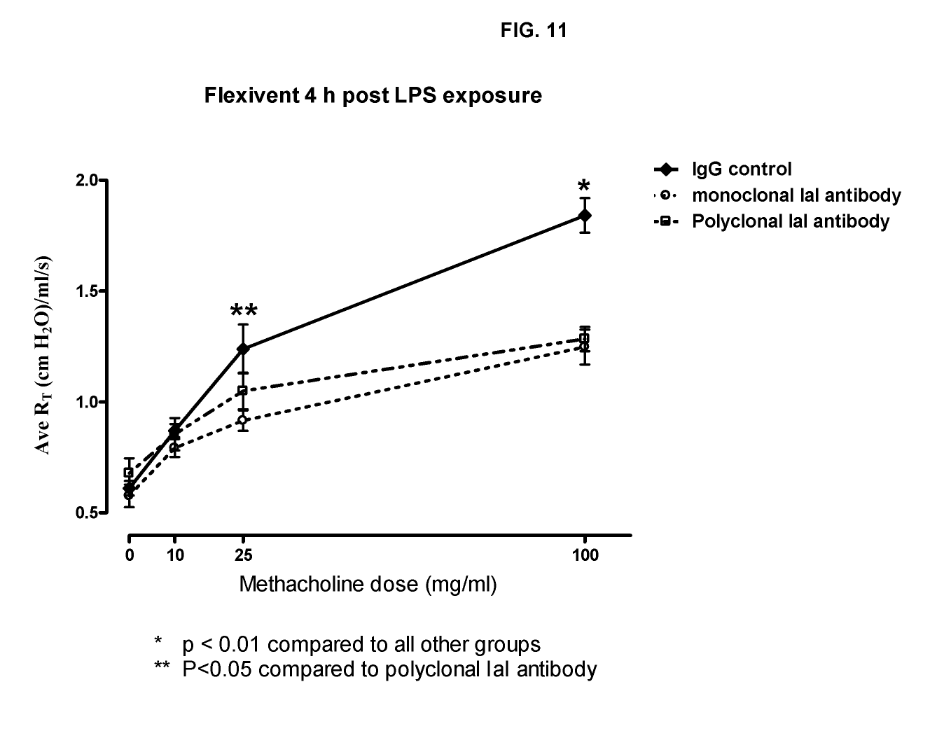

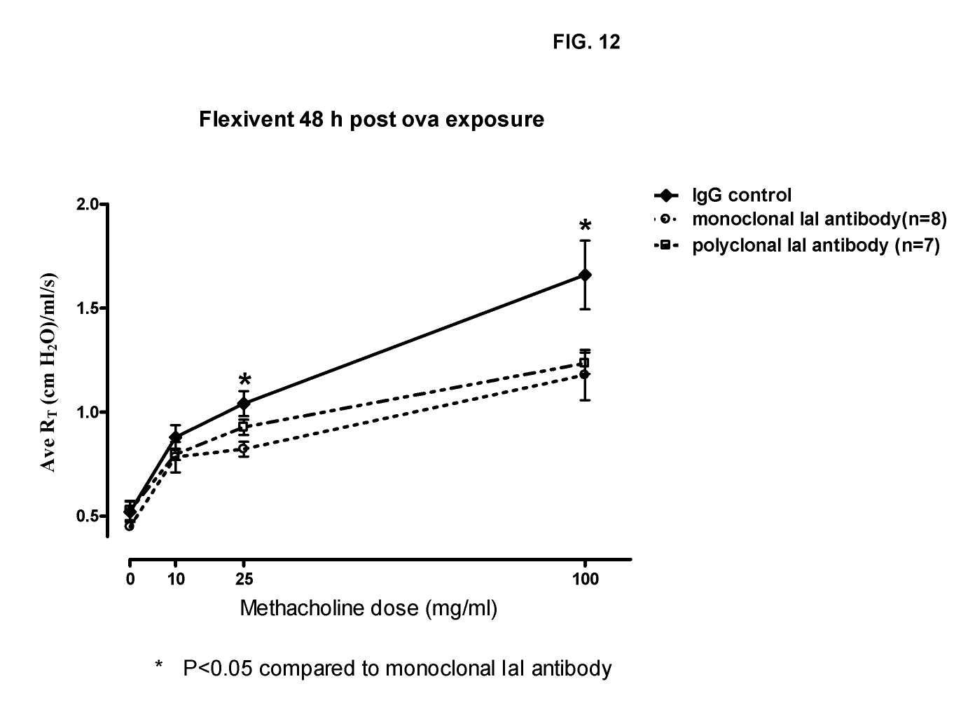

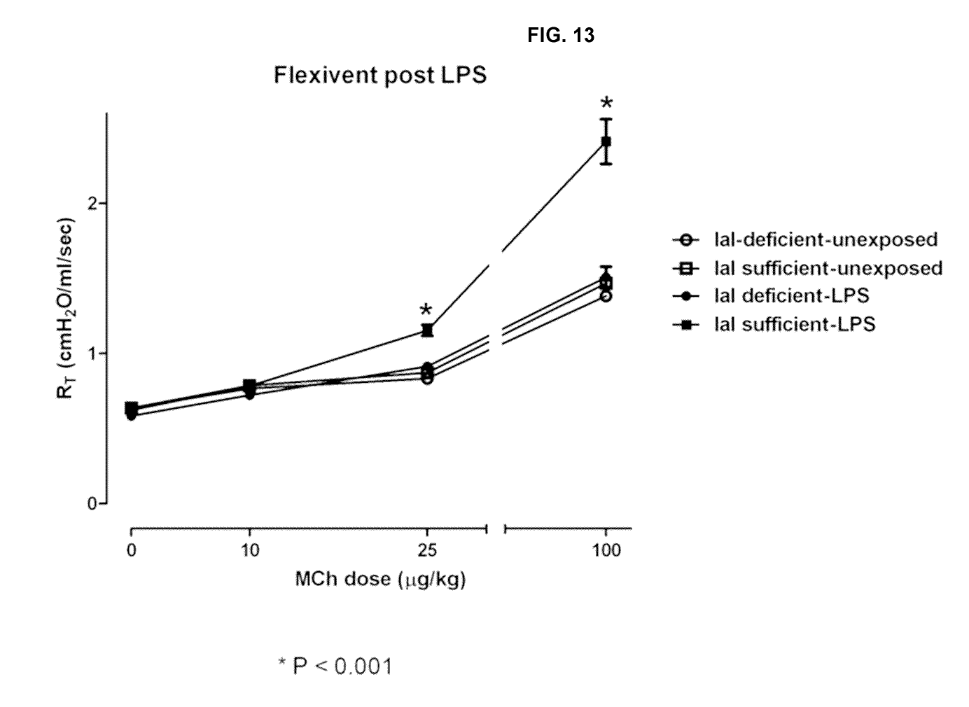

In animals deficient for IaI, AHR is significantly reduced following exposure to LPS, relative to wild-type animals (FIG. 13). As shown in FIGS. 10-12, administration of either a monoclonal antibody to IaI or a polyclonal antibody to IaI, significantly decreased airway hyperresponsiveness following exposure to either ozone (FIG. 10), LPS (FIG. 11) or OVA (FIG. 12), relative to an IgG antibody control. A significant decrease in airway hyperresponsiveness was observed at all doses of methacholine in the ozone model, and a significant decrease in airway hyperresponsiveness was observed at methacholine doses of 25 and 100 mg/ml for the LPS- and OVA-exposed animals Importantly, a decrease in AHR was observed even after significant delays in administration of IaI antibody (four hours after LPS exposure, 24 hours after ozone exposure and 48 hours after OVA exposure), which is representative of the timing of treatment of airway disease in human subjects. These results demonstrate that functional blockade of IaI inhibits airway constriction in three animal models of airway inflammation, indicating that IaI is a viable therapeutic target for the treatment of human airway diseases.

### Example 9

### Functional Blockade of IaI in a Guinea Pig Model of AHR

This example describes a guinea pig model that can be used to evaluate the effect of IaI inhibitors, such as IaI-specific antibodies, on the development of AHR in response to ozone. Guinea pig models of AHR have been described in the art (see, for example, Verhein et al., Am J Respir Cell Mol Biol 39:730-738, 2008).

Pathogen-free Dunkin-Hartley guinea pigs (Elm Hill Breeding Labs, Chelmsford, Mass.) are exposed to 2 ppm ozone or filtered air for approximately 4 hours (Yost et al., J Appl Physiol 87:1272-1278, 1999). IaI antibody (for example, MAb 69.26, anti-IaI scFv, or polyclonal IaI antibody), diluted in PBS, is administered intraperitoneally. PBS-injected animals serve as controls. IaI antibody can be administered either before exposure to ozone, during exposure, or following exposure, such as 24 hours after exposure. One to three days following exposure to ozone, animals are anesthetized with urethane (1.9 g/kg, intraperitoneally) and evaluated for AHR according to standard procedures (see Verhein et al., Am J Respir Cell Mol Biol 39:730-738, 2008).

### Example 10

### Functional Blockade of IaI in Chronic Models of Airway Disease

This example describes animal models that can be used to represent chronic airway disease, such as asthma or COPD. Animal models of chronic airway disease have been described in the art (see, for example, Savov et al., Am J Physiol Lung Cell Mol Physiol 283(5):L952-962, 2002).

### Chronic Ozone Exposure

C57BL/6J mice are exposed to either Hepa-filtered air or ozone. Animals are housed in cages with low-endotoxin bedding, and given water and chow ad libitum. Animals are exposed to 0.3 ppm ozone for 72 hours. Exposures are performed in 55-liter Hinner chambers with individual animal slots. Air at 20-22° C. and 50-60% relative humidity is supplied at 20 exchanges per hour. Ozone is generated by directing 100% O₂ through a UV light generator, and mixed with air supply to the chamber. Chamber ozone concentration is monitored continuously with a UV light photometer (1003AH, Dasibi, Glendale, Calif.). IaI antibody is administered either daily during exposures, or once at the end of the exposure. Mice are subjected to invasive AHR measurement and euthanized, and subsequently lung tissue is collected.

### LPS Exposure

Lyophilized, reconstituted LPS (Escherichia coli serotype 0111:B4, Sigma, St. Louis, Mo.) is used. LPS aerosol was generated as previously described (Savoy). Briefly, a six-jet Atomizer (Model 9306, TSI Inc., Shoreview, Minn.) is used at a constant pressure of 35 psi. Mice are exposed for 2.5 hours (acute exposure), or for 2.5 hours per day, 5 days per week, for one to four weeks (chronic exposure). LPS concentrations are determined by sampling the total chamber outflow, using the quantitative chromogenic Limulus amebocyte lysate (LAL) assay (QCL-1000; Whittaker Bioproducts, Walkersville, Md.). The concentrations of LPS aerosol (LAL assay) in these experiments are 6-8 μg/m³. IaI antibody is administered either daily during exposures, or once at the end of the exposure. Mice are subjected to invasive AHR measurement and euthanized, and subsequently lung tissue is collected.

### Example 11

### Inhibition of IaI Binding to Hyaluronan

This example demonstrates that IaI-specific antibodies that ameliorate AHR inhibit binding of IaI to hyaluronan. The data described in Example 8 above demonstrate that functional blockade of IaI is effective several hours after environmental exposure, independently of inflammatory parameters in both Th1 (LPS) and Th2 (OVA) models of murine airway inflammation. Furthermore, because IaI-deficient mice have similar airway resistance as wild-type mice in the naïve state, and because the IaI-specific antibodies do not affect airway resistance in naïve mice, it is likely that IaI is not influencing baseline airway tone. It was therefore hypothesized that IaI inhibition affects a common final pathway of airway hyperresponsiveness. Such a pathway may involve hyaluronan binding to smooth muscle cells.

Hyaluronan binding to cells has been shown to lead to activation of the neural Wiskott-Aldrich syndrome protein (N-WASP), and ultimately promote actin polymerization via the Arp2/3 complex (Bourguignon et al., J. Biol. Chem. 282(2):1265-1280, 2007). N-WASP activation and Arp2/3 complexing has been shown to be necessary for AHR (Zhang et al., Am. J. Physiol. 288(5):C1145-1160, 2005). It was therefore investigated whether IaI blockade can affect this pathway.

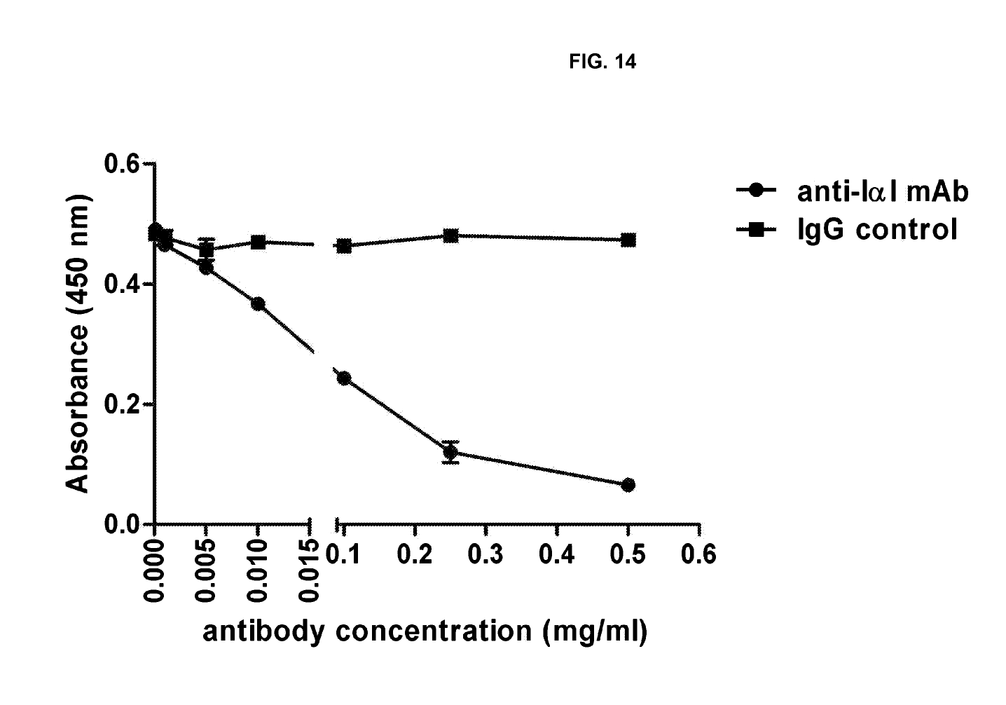

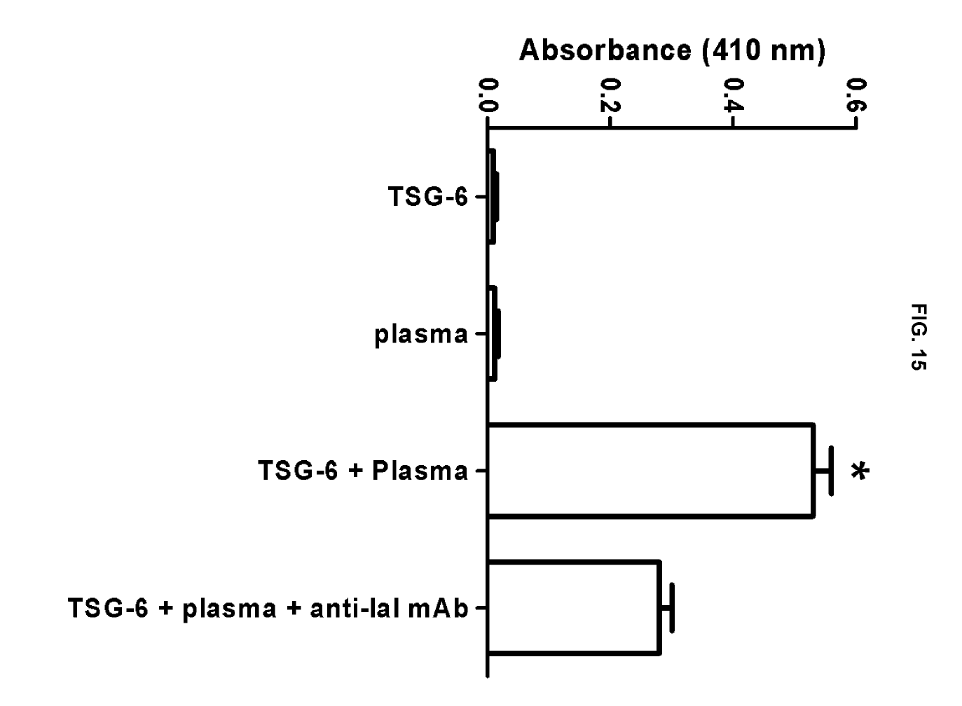

To determine whether an IaI-specific monoclonal antibody can block binding of IaI to hyaluronan, a competition ELISA was performed. Plates were coated with hyaluronan and incubated with IaI and increasing concentrations of anti-IaI antibody or an IgG control antibody. As shown in FIG. 14, a significant decrease of IaI-hyaluronan binding was observed with the anti-IaI antibody, and binding decreased as the concentration of anti-IaI antibody increased. No alteration in IaI-hyaluronan binding was observed with the control antibody. Next, the effect of anti-IaI antibody on IaI heavy chain-hyaluronan binding was evaluated. The heavy chain transfer from IaI in plasma (1:1000) to hyaluronan was measured by ELISA. Anti-IaI antibody was used at a final dilution of 1:1000. Diluted plasma was incubated with the antibody overnight at 4° C. and the heavy chain transfer was performed thereafter by addition of TSG-6 and transfer into hyaluronan plates. As shown in FIG. 15, the anti-IaI antibodies specifically inhibit hyaluronan binding to IaI heavy chains.

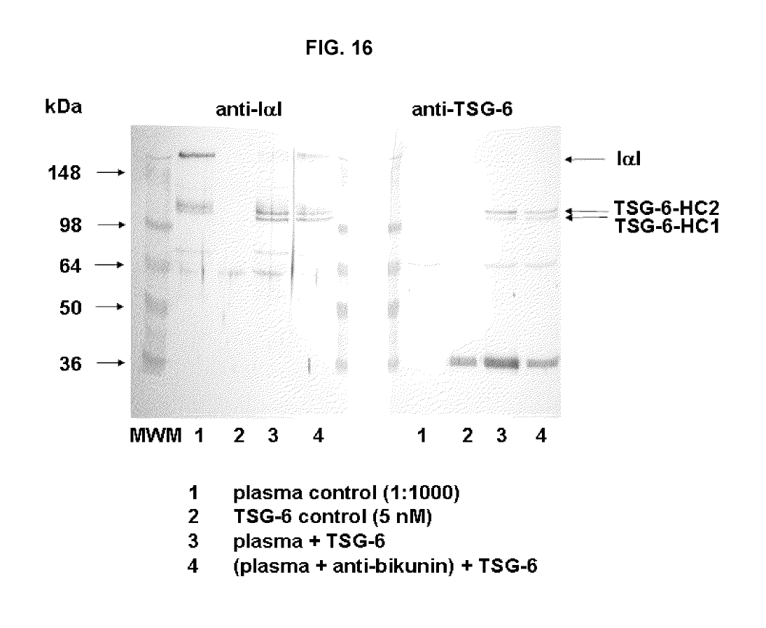

It is known that hyaluronan binding to IaI heavy chains is dependent on the creation of an IaI heavy chain-TSG-6 complex (Sanggaard et al., J. Biol. Chem. 283(27):18530-18537, 2008). Therefore, the effect of pre-incubation of plasma with monoclonal anti-IaI antibody on the transfer of heavy chains from IaI to TSG-6 was evaluated. Plasma was incubated with TSG-6 with or without IaI-specific antibody, and IaI-TSG-6 complexes were detected using appropriate antibodies in a western blot (FIG. 16). In the presence of anti-IaI antibody (anti-bikunin), there is a significantly less dense TSG-6-HC2 band (compare lane 3 and lane 4 of FIG. 16). There is also significantly more free IaI in the presence of anti-IaI antibody (see left panel of FIG. 16), indicating that IaI is not being consumed in the binding reaction. These results demonstrate that the IaI-specific antibodies inhibit the generation of the heavy chain-TSG-6 complex.

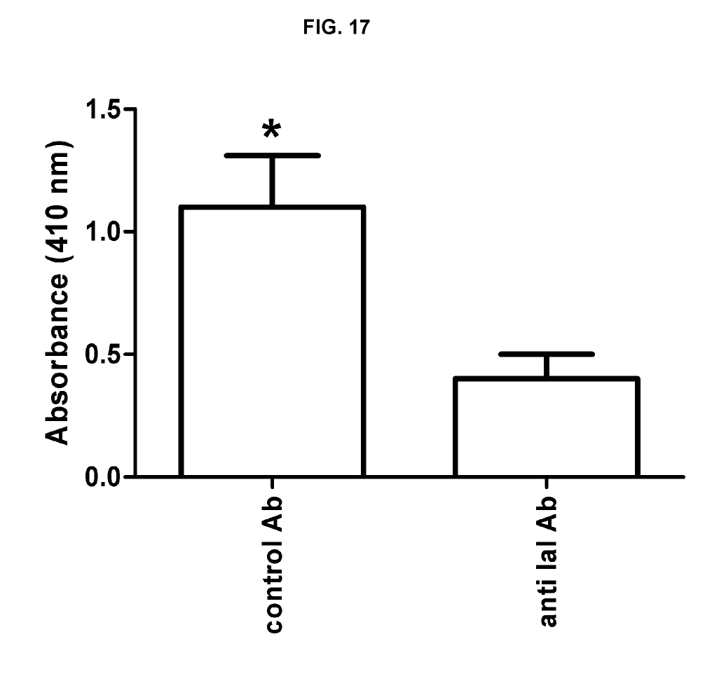

Having established that the antibodies indeed inhibit hyaluronan-IaI binding, studies were performed to determine whether IaI antibodies also inhibit N-WASP-Arp2/3 complexing in vivo. Mice were exposed to inhaled endotoxin, then treated with the monoclonal anti-IaI antibody or IgG control. Following sacrifice, tracheas and main-stem bronchi were removed and homogenized, and the homogenates were subjected to immunoprecipitated for N-WASP. A western blot using anti-Arp2/3 antibody was carried out to detect co-immunoprecipitation of N-WASP and Arp2/3. FIG. 17 shows the densitometry results for the co-immunoprecipitation experiment. Anti-IaI treated mice showed significantly less Arp2/3, indicating decreased N-WASP-Arp2/3 complex formation. Thus, these results suggest that functional blockade of IaI ameliorates AHR by blocking IaI-hyaluronan binding.

### Example 12

### IaI Promotes AHR in Human Asthma

This example describes the finding that levels of IaI are significantly increased in bronchoalveolar lavage fluid (BALF) from asthmatic human subjects following exposure to endotoxin or dust mite antigen, compared with non-asthmatic subjects. The data described in this example further demonstrates that anti-IaI antibody inhibits the development of AHR in mice administered BALF from human asthmatic subjects.

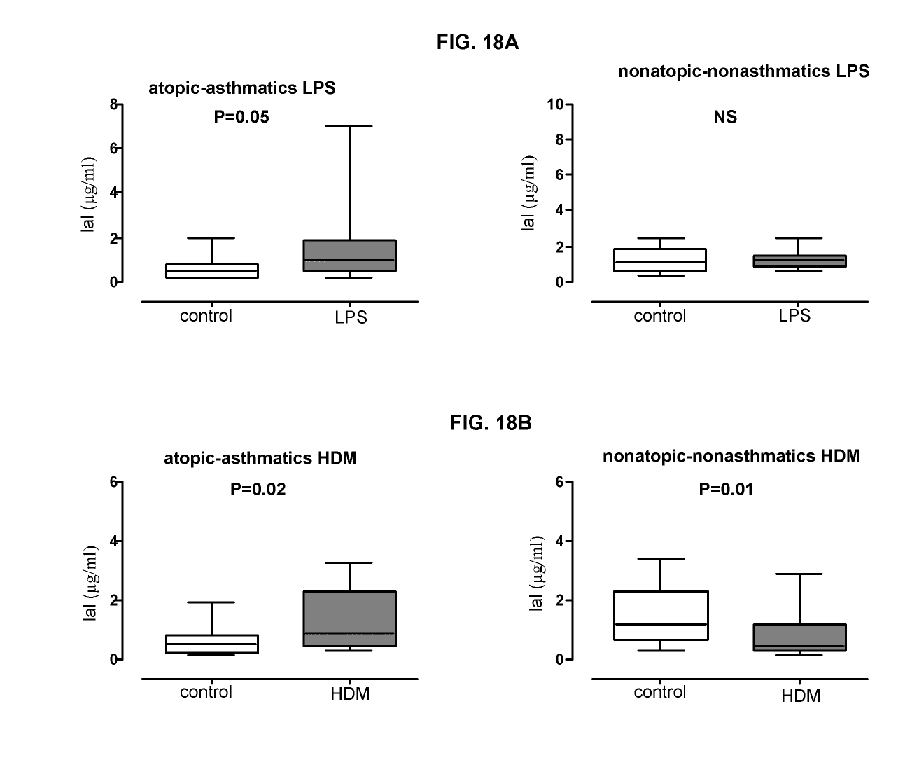

To determine whether IaI is important in human asthma, levels of IaI were measured in BALF of either asthmatic subjects or non-asthmatic controls, after topical (bronchial) instillation of endotoxin (LPS), house dust mite antigen extract (HDM) or a control solution (Hanks balanced salt solution). It was determined that asthmatic individuals had significantly increased IaI levels compared to their baseline levels after exposure to LPS (FIG. 18A) or HDM (FIG. 18B). In contrast, non-asthmatic individuals exhibited no appreciable change in BALF IaI levels.

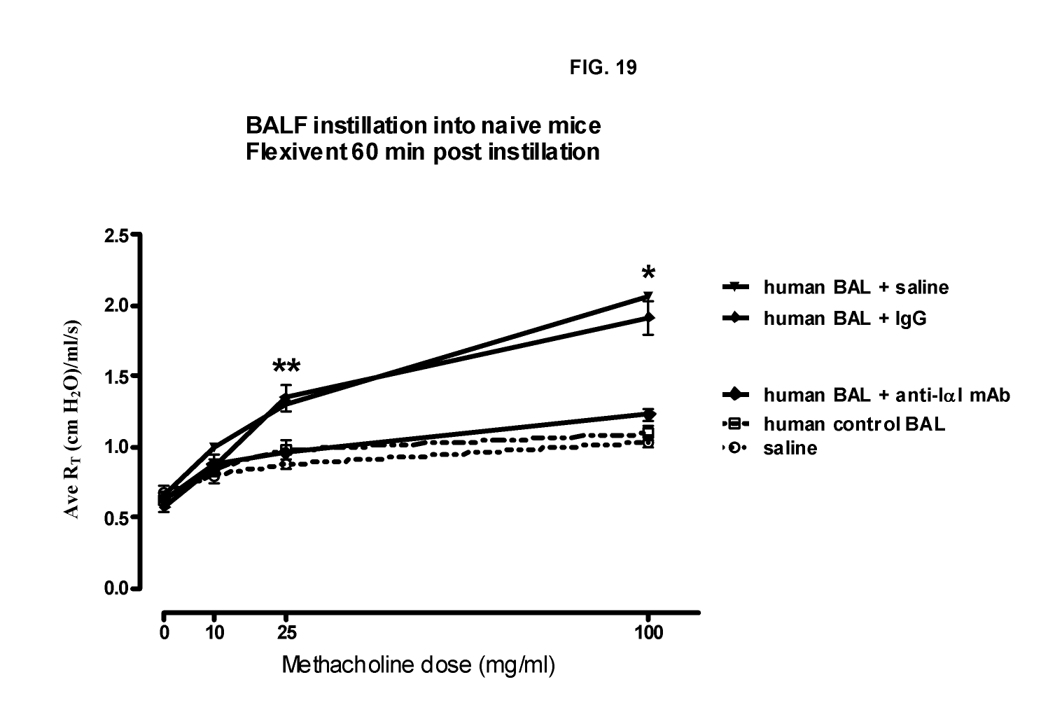

Next, studies were performed to determine if IaI is important in the development of AHR in human asthma. BALF was pooled from exposed or unexposed airways from atopic-asthmatic individuals and nonatopic-nonasthmatic subjects. Pooled BALF was instilled intratracheally into naïve mice, with either an IaI antibody, a control antibody, or vehicle. Control mice received BALF from sham-exposed bronchi, or normal saline. Sixty minutes after instillation of BALF, AHR was measured using flexiVent™, as described in the Examples above. The results, shown in FIG. 19, show that BALF from these airways induced AHR in mice, but not when IaI antibody was administered. These results demonstrate that IaI is important in the pathogenesis of AHR in human asthma.

In view of the many possible embodiments to which the principles of the disclosed subject may be applied, it should be recognized that the illustrated embodiments are only preferred examples of the disclosure and should not be taken as limiting the scope of the invention. Rather, the scope of the invention is defined by the following claims.

1. A method of treating an airway disease or disorder in a subject, comprising (i) selecting a subject in need of treatment; and (ii) administering to the subject a therapeutically effective amount of an inhibitor of inter-alpha-trypsin inhibitor (IaI), wherein the inhibitor of IaI is an antibody, polypeptide, small molecule or antisense compound, thereby treating the airway disease or disorder.

2. The method of claim 1, wherein the airway disease or disorder is a chronic disease or disorder.

3. The method of claim 2, wherein the chronic disease or disorder is selected from asthma, chronic obstructive pulmonary disease, cystic fibrosis, obliterative bronchiolitis, diffuse panbronchiolitis and cryptogenic organizing pneumonia.

4. The method of claim 1, wherein the airway disease or disorder is an acute disease or disorder.

5. The method of claim 4, wherein the acute disease or disorder is selected from exercise-induced asthma, respiratory infection, acute bronchiolitis, airway hyperresponsiveness, pollution-induced airway injury, chemical-induced airway injury and ventilation-induced airway injury.

6. The method of claim 1, wherein the inhibitor of IaI is an antibody.

7. The method of claim 6, wherein the antibody is a monoclonal antibody that specifically binds IaI.

8. The method of claim 7, wherein the monoclonal antibody is a humanized antibody.

9. The method of claim 6, wherein the antibody is a polyclonal antibody.

10-12. (canceled)

13. The method of claim 1, wherein the antisense compound is an antisense oligonucleotide, siRNA, miRNA, shRNA or ribozyme that specifically hybridizes with a nucleic acid molecule encoding IaI.

14. The method of claim 13, wherein the antisense compound specifically hybridizes with a nucleic acid molecule encoding the IaI light chain bikunin.

15. The method of claim 13, wherein the antisense compound specifically hybridizes with a nucleic acid molecule encoding one or more of the IaI heavy chains.

16. The method of claim 1, wherein administration of the inhibitor comprises local delivery to the airway.

17. The method of claim 16, wherein local delivery comprises aerosol delivery.

18. The method of claim 17, wherein the inhibitor is administered by aerosol using an inhaler or a nebulizer.

19. The method of claim 18, wherein the inhaler is a dry powder inhaler or a metered-dose inhaler.

20-21. (canceled)

22. A method of preventing or reducing airway hyperresponsiveness (AHR) in a subject, comprising administering to the subject a therapeutically effective amount of an inhibitor of inter-alpha-trypsin inhibitor (IaI), wherein the inhibitor of IaI is an antibody, polypeptide, small molecule or antisense compound, thereby preventing or reducing AHR.

23. The method of claim 22, wherein the subject suffers from asthma or COPD.

24. The method of claim 22, wherein AHR is triggered by an environmental trigger, a chemical trigger, exertion or stress.

25. (canceled)

26. The method of claim 24, wherein the IaI inhibitor is administered prophylactically prior to exposure to the trigger.

27-28. (canceled)
